(19) United States
(12) Patent Application Publication
(10) Pub. No.: US 2023/0041111 A1
(43) Pub. Date: Feb. 9, 2023

(54) SYSTEMS AND METHODS FOR INTRAOPERATIVE SURGICAL SCOPE CLEANING

(71) Applicant: Stryker Corporation, Kalamazoo, MI (US)

(72) Inventor: Jared COFFEEN, Hollister, CA (US)

(73) Assignee: Stryker Corporation, Kalamazoo, MI (US)

(21) Appl. No.: 17/817,653

(22) Filed: Aug. 4, 2022

Related U.S. Application Data
(60) Provisional application No. 63/229,969, filed on Aug. 5, 2021.

(51) Int. Cl. A61B 90/70; A61B 1/313; A61B 1/12

(52) U.S. Cl. CPC A61B 90/70; A61B 1/3132; A61B 1/126; A61B 1/127

(57) ABSTRACT

A system for supplying insufflation gas for a surgical procedure includes first and second insufflation gas inlets for receiving insufflation gas from at least two insufflation gas supply tanks located in an operating room; an insufflation gas outlet for providing a flow of insufflation gas supplied via the first and second insufflation gas inlets; and a valve system configured to automatically switch from insufflation gas supply via the first insufflation gas inlet to insufflation gas supply via the second insufflation gas inlet to maintain insufflation gas flow at the insufflation gas outlet.

### CROSS-REFERENCE TO RELATED APPLICATIONS

This application claims the benefit of U.S. Provisional Application No. 63/229,969, filed Aug. 5, 2021, the entire contents of which are hereby incorporated by reference herein.

### FIELD

The present disclosure relates generally to endoscopic surgery, and more particularly to cleaning a surgical scope, such as during a surgical procedure.

### BACKGROUND

Although laparoscopic surgery has been performed going back as far as 1901, it became more widespread upon the introduction of the rigid laparoscope with a rod lens optical train and glass fiber optic illumination in the mid 1980's. Since then, laparoscopic surgery has evolved into the standard of care for many types of abdominal surgery.

Since a surgeon is dependent on the image provided by the laparoscope, the surgeon's performance is impaired if the lens at the distal end of the laparoscope is not kept clean while in the surgical cavity. For example, the surgeon can have difficulty viewing the surgical field when any of the following occurs: the surface temperature of the lens is lower than the temperature of the surgical cavity and condensation forms on the lens, which is referred to as “scope fogging”; the lens touches tissue in the surgical cavity during the course of the surgery and becomes soiled with fat, blood, pieces of tissue, bile, etc., which is referred to as “scope smudging”; fluids splash or squirt at the laparoscope during the surgery and accumulate on the lens, such as blood from a perforated artery, irrigation fluid while washing the surgical site with pressurized saline, etc., which is also referred to as “scope smudging”; and the laparoscope is passed through a trocar in order to enter the surgical cavity and the lens touches blood, fat, pieces of tissue, or lubricant from the seals of the trocar, which is also referred to as “scope smudging”.

Many attempts have been made to address the problems of lens fogging and scope smudging. However, these attempts have been largely unsuccessful and surgeons continue to remove the laparoscope from the surgical cavity for cleaning and then subsequently re-insert the laparoscope into the surgical cavity to continue the surgery. Often, re-insertion into the surgical cavity through a trocar smudges the scope again, and the cleaning process must be repeated until the surgeon is able to obtain a clear image of the surgical cavity.

Attempts to solve scope smudging and fogging have often been ineffective for several reasons. Designs with lens-cleaning features built into the scope itself have the benefit of not requiring the surgeon to remove the scope from the surgical cavity during surgery but can substantially complicate the design of the scope and make cleaning and sterilizing the scope difficult and can affect the scope's reliability and useful life. Designs having a mechanism for mechanically cleaning the scope, such as wipers or sponges, have difficulty keeping the mechanism clean and dry enough to be effective at cleaning the lens over the course of a surgery. Such designs may also require the surgeon to move the scope back and forth past the cleaning mechanism, which can distract the surgeon from the surgery.

Designs that have a sheath for preventing the lens from being contacted by fluids and debris substantially complicate the process of cleaning the lens should the lens be smudged because access to the lens is made more difficult, often requiring removal of the sheath to properly clean the lens. Designs that include a sheath that, together with the outer surface of the scope, forms a lumen for fluid or gas to pass through for cleaning the lens can generally only be configured to work with a particular make and model of laparoscope because the fit between the scope and the sheath is critical. The manufacturing tolerances of the scope and the sheath as well as the fact that the mating surface of one or the other over time will get damaged due to reprocessing by hospital staff can make such design impractical.

Designs that have a film that protects the lens from making contact with the fluids and tissue during surgery can suffer from a number of drawbacks. Anything positioned in front of the lens of the scope will cause some level of image degradation. The film may not always be able to seal perfectly and prevent the lens from getting smudged and fluid from penetrating the sheath and remaining there, which can cause any new film that is advanced in front of the lens to also become smudged. Further, the film may not help prevent fogging, which means that the scope must be properly warmed directly before installing the sheath and inserting into the surgical cavity. If the scope is removed during the surgery for any reason, it must be warmed again before being reinserted into the surgical cavity or else it will get fogged again.

Designs that spray a cleaner at the lens and suction the waste away have not been successful in laparoscopic surgery since it is often difficult to rely on a suction flow to always pull the tiny drops of fluid across the lens for removal due to the surface tension between the glass and the fluid droplets.

### SUMMARY

According to an aspect, a system for supplying insufflation gas, such as for a surgical procedure, includes first and second insufflation gas inlets for receiving insufflation gas from at least two insufflation gas supply tanks located in an operating room; an insufflation gas outlet for providing a flow of insufflation gas supplied via the first and second insufflation gas inlets; and a valve system configured to automatically switch from insufflation gas supply via the first insufflation gas inlet to insufflation gas supply via the second insufflation gas inlet to maintain insufflation gas flow at the insufflation gas outlet.

Optionally, the system further includes a sensor system for detecting at least one pressure, at least one flow rate, or at least one of both pressure and flow rate that is associated with the at least two insufflation gas supply tanks, wherein the valve system includes at least one valve and a control system configured to control the at least one valve to switch from insufflation gas supply via the first insufflation gas inlet to insufflation gas supply via the second insufflation gas inlet.

Optionally, the valve system comprises a two-way valve in fluid communication with the first and second insufflation gas inlets.

Optionally, the valve system comprises at least two one-way valves.

Optionally, the sensor system comprises at least one flow sensor for detecting an insufflation gas flow rate associated with at least one of the first and second insufflation gas inlets, and wherein the control system controls the valve system to actuate the at least one valve until the insufflation gas flow rate is sufficiently reduced.

Optionally, the control system is configured to switch from insufflation gas supply via the first insufflation gas inlet to insufflation gas supply via the second insufflation gas inlet upon determining that the at least one pressure or the at least one flow rate is below a predetermined threshold.

Optionally, the control system is configured to provide a notification indicative of a depletion of at least one of the at least two insufflation gas supply tanks.

Optionally, the notification is provided on a display of the system.

Optionally, the control system is configured to transmit the notification to an external system via a network connection.

Optionally, the notification provides an indication of which of the at least two insufflation gas supply tanks is depleted.

Optionally, the valve system comprises a valve that comprises two inlets, and the valve automatically actuates based on a pressure differential between the two inlets.

Optionally, the system is portable.

Optionally, the outlet is configured for fluidly connecting to an insufflation gas inlet of an insufflator.

Optionally, the system is an insufflator.

According to an aspect, a method for supplying insufflation gas for a surgical or nonsurgical procedure includes supplying insufflation gas from a first insufflation gas supply tank located in an operating room to a field, such as a surgical field; and automatically switching from supply by the first insufflation gas supply tank to supply by a second insufflation gas supply tank based on at least one of a reduction in pressure of the first insufflation gas supply tank and a reduction in flow rate from the first insufflation gas supply tank.

Optionally, the method includes providing a notification of a depletion of the first insufflation gas supply tank.

Optionally, the notification indicates which of the at least two insufflation gas supply tanks is depleted.

Optionally, the method includes monitoring a pressure of the first insufflation gas supply tank and automatically actuating a valve to switch the supply in response to determining that a pressure of the first insufflation gas supply tank is below a predetermined threshold.

Optionally, the supply is automatically switched by a valve that automatically actuates based on a pressure differential.

Optionally, the method further includes monitoring an amount of insufflation gas provided to the, e.g. surgical, field and detecting an insufflation gas supply leak by comparing the amount of insufflation gas provided to the, e.g. surgical, field to a drop in pressure of at least one of the first and second insufflation gas supply tanks.

According to an aspect, an apparatus for cleaning a surgical scope includes a sheath for removably receiving a tube of the surgical scope, the sheath comprising a wall defining a channel for receiving the tube and a conduit that defines a fluid flow path; a nozzle located at a distal end of the distal portion of the wall and configured for directing a fluid flow across a lens of the surgical scope to clean the lens; and a first inlet for connecting a gas supply for supplying a gas flow to the fluid flow path and a second inlet for connecting a liquid supply for supplying a liquid flow to the fluid flow path.

Optionally, the apparatus further comprises a valve for fluidly connecting and disconnecting the first and second inlets, respectively, to the fluid flow path.

Optionally, the valve actuates automatically based on a pressure differential between the first and second inlets.

According to an aspect, a method for cleaning a surgical scope includes inserting the surgical scope into a sheath of a surgical scope cleaner; flowing a liquid through a conduit of the sheath and spraying a lens of the surgical scope with the liquid via a nozzle of the surgical scope cleaner; and flowing a gas through the conduit of the sheath and blowing the lens of the surgical scope with the gas via the nozzle of the surgical scope cleaner to remove the liquid from the lens.

Optionally, the method includes closing a gas supply pathway while flowing the liquid through the conduit and closing a liquid supply pathway while flowing the gas through the conduit.

Optionally, the liquid and gas supply pathways automatically close and open based on a pressure differential between the liquid and gas supply pathways.

According to an aspect, a system for supplying insufflation gas for a surgical procedure includes a first gas supply configured to provide a gas at a first pressure for insufflation of a patient; a second gas supply configured to supply gas at a second pressure that is higher than the first pressure for pressurizing a liquid reservoir that supplies liquid for irrigation during a surgical procedure; and a third gas supply configured to supply gas at a third pressure that is higher than the second pressure for supply gas to one or more surgical tools.

According to an aspect, a system for supplying one or more fluids during a surgical procedure includes a receptacle configured to accept multiple different configurations of tube sets; and a fluid delivery system configured to supply one or more one or more fluids to a connected tube set based on a configuration of the connected tube set.

According to an aspect, a tube set for supplying fluid flow to a surgical field includes at least one fluid supply tube for supplying a fluid to a surgical field for cleaning an endoscope of an endoscopic imager; and a fiber optic light cable attached to the at least one fluid supply tube for providing illumination light to the endoscopic imager.

According to an aspect, a fluid supply line for supplying a fluid to a surgical device includes a shut-off at an outlet end of the tube that is configured to automatically shut off flow out of the tube when the tube is disconnected from the device.

According to an aspect, a fluid supply system for supplying insufflation gas for a surgical procedure includes a first gas outlet for supplying an insufflating gas to an insufflating gas delivery device located in a surgical cavity; a second gas outlet for supplying the insufflating gas to a surgical scope cleaner located in the surgical cavity; a valve system configured to supply a first flow of the insufflating gas via the first outlet and a second flow of the insufflating gas via the second outlet; a controller configured to control the valve system to insufflate the surgical cavity by continuously supplying the first flow of insufflating gas to the insufflating gas delivery device located in a surgical cavity and, simultaneously, continuously supplying the second flow of insufflating gas to the surgical scope cleaner.

According to an aspect, a system for cleaning a surgical scope of an endoscopic imager includes a control system communicatively connected to an apparatus for supplying fluids to a surgical scope cleaner, the control system configured to: receive one or more images of a surgical field generated by the endoscopic imager, detect a deposit on a lens of the surgical scope by analyzing the one or more images, provide a notification to a user indicating that a deposit on the lens has been detected, receive a user command to execute a cleaning sequence, and in response to receiving the user command, wait a predetermined period of time and send a command to the apparatus to execute the cleaning sequence.

According to an aspect, a system for cleaning a surgical scope of an endoscopic imager includes a control system communicatively connected to an apparatus for supplying fluids to a surgical scope cleaner, the control system configured to: automatically execute a cleaning operation for cleaning the surgical scope cleaner based on at least one user settable parameter, wherein the at least one user settable parameter comprises: a length of time a washing fluid is supplied to the apparatus, a length of time a gas is supplied to the apparatus, a pressure of the washing fluid supplied to the apparatus, a pressure of the gas supplied to the apparatus, and whether to include a washing step in addition to a gas blowing step in the cleaning sequence.

### BRIEF DESCRIPTION OF THE FIGURES

Features will become apparent to those of ordinary skill in the art by describing in detail exemplary embodiments with reference to the attached drawings in which:

FIGS. 1A and 1B illustrate a surgical scope cleaner mounted on a surgical scope, According to various aspects;

FIG. 1C illustrates the distal end of a scope cleaner mounted on a surgical scope, According to various aspects;

FIG. 1D is a cross-section of a sheath of a scope cleaner illustrating liquid and gas conduits, According to various aspects;

FIG. 1E is a cross-section of distal portions of a scope cleaner and surgical scope 150, According to various aspects;

FIGS. 1F and 1G illustrate an alternative arrangement for locating the nozzle head outside of the field of view of the surgical scope, According to various aspects;

FIG. 1H illustrates an arrangement of a scope cleaner in which the sheath is not configured to angle away from the tube;

FIGS. 1I and 1J illustrate operation of a surgical scope cleaner, According to various aspects;

FIG. 2 is a cross section of a portion of a scope cleaner that is configured for warming and for providing a steady stream of gas to a surgical scope, According to various aspects;

FIG. 3 is a block diagram of an apparatus for managing fluid flow into and out of a surgical field, According to various aspects;

FIGS. 4A-4D illustrate a fluid supply apparatus and connector for connecting multiple fluid lines to the apparatus, According to various aspects;

FIGS. 5A and 5B illustrate a fluid supply apparatus and tube set connector in which a liquid supply reservoir, for supplying the liquid for the scope cleaner, is incorporated into the connector, According to various aspects;

FIGS. 6A and 6B illustrate a fluid supply apparatus and tube set connector in which a pump is incorporated into the connector for pumping liquid from an external liquid supply reservoir to the scope cleaner, According to various aspects;

FIG. 7 illustrates the use of a tube set in a surgical field, According to various aspects;

FIGS. 8A and 8B illustrate the connection of tube set to a fluid supply management apparatus, According to various aspects;

FIG. 9 illustrates a method for supplying fluid to a, e.g. surgical, field, According to various aspects;

FIG. 10 illustrates a method for cleaning a surgical scope, particularly while the surgical scope is inserted in a, e.g. surgical, cavity, According to various aspects;

FIG. 11A illustrates an scope with integrated cleaning, According to various aspects;

FIG. 11B illustrates a perspective view of a cross-section of a portion of a shaft of a scope with integrated cleaning, According to various aspects;

FIGS. 11C and 11D are perspective and cross-sectional views, respectively, of a distal portion of a scope with integrated cleaning, According to various aspects;

FIG. 11E is a cross section of a proximal portion of a shaft and a portion of a main body of a scope with integrated cleaning, According to various aspects;

FIG. 12 illustrates a method for automatically detecting deposits on a lens of a surgical scope and sending a command to a connected apparatus to initiate a cleaning sequence for the surgical scope, according to various aspects;

FIGS. 13A and 13B illustrate an exemplary surgical scope cleaner in which the sheath includes a single conduit that provides a single pathway for both liquid and gas to flow;

FIG. 14 is a block diagram of an exemplary fluid delivery system that automatically switches gas supply sources to ensure a continuous supply of gas to a surgical field;

FIG. 15 illustrates an exemplary display that indicates the statuses of two gas supply tanks;

FIG. 16 illustrates an exemplary method for supplying gas, such as insufflation gas, particularly for a surgical procedure; and

FIG. 17 illustrates an exemplary quick-connect arrangement for a supply tube that shuts off fluid flow when the tube is disconnected.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described below are systems and methods, according to various aspects, for cleaning a surgical scope, particularly for cleaning a surgical scope during a surgical or nonsurgical procedure with minimal interruption of the surgical or nonsurgical procedure. According to various aspects, a surgical scope cleaner includes a sheath that slides over the surgical scope and includes at least one nozzle for flushing the surface of the lens with a spray of a liquid such as saline and then blowing the lens with a burst of a gas such as carbon dioxide. Conduits running along the sheath lead from input ports at an end of the cleaner that can be connected to pressurized liquid and gas sources via a tube set. The sheath can be configured to slide over a standard size scope and to fit through the lumen of a standard size trocar. The surgical scope with mounted scope cleaner can be inserted or pre-inserted through a trocar into a cavity and the cleaner can be used to clean the scope during the procedure with minimal disruption to the procedure. The surgical scope with mounted scope cleaner can be inserted or pre-inserted through a trocar into the surgical cavity, and the cleaner can be used to clean the scope during the surgical procedure with minimal disruption to the procedure.

The at least one nozzle incorporated into the end of the sheath can point towards the lens of the scope to spray the cleaning liquid and blow gas with a high velocity directly at the lens. The at least one nozzle can be configured so that the high-velocity liquid spray clears off the entire surface of the lens—i.e., pressure washing the lens. The burst of gas can be provided after the liquid spray is complete to blow the surface of the lens dry and can also be provided at the same time as the liquid spray to enhance the liquid spray, increasing its velocity and hence its cleaning power. According to various aspects, the sequence of the liquid spray and the gas burst, as well as the length of time they are activated, can be controlled by electromechanical valves in a fluid management system to which the scope cleaner is connected.

Optionally, the liquid and gas used for the scope cleaner are saline and carbon dioxide, which are used in most laparoscopic surgeries—the saline is often used to flush or irrigate when needed during surgery and carbon dioxide is often used to insufflate (or distend) the abdomen. Saline has been shown to be able to sufficiently clean blood, fat, and tissue debris from the lens of scopes used in surgery. Thus, surgical scope cleaning, According to various aspects, can be incorporated into existing surgical systems.

According to various aspects, a fluid management system to which the scope cleaner is connected can also be used to manage other fluids used in a surgical procedure. A fluid management system that provides, for example, carbon dioxide to the scope cleaner can also serve as an insufflator, providing the carbon dioxide to insufflate the surgical cavity. Optionally, the pressurized carbon dioxide gas that is received and regulated by the system for insufflation can also be used to pressurize the saline for the lens flushing and to blow the lens dry after the flushing cycle. Thus, scope cleaning can be provided without having to add an additional piece of equipment to the operating room.

According to various aspects, the scope cleaner can be integrated into an insufflator tube set, which can help reduce clutter in the sterile field where the surgeon is operating. Clutter caused by the many hoses and wires that are attached to instruments in the sterile field and to control units and supply lines from outside the sterile field can restrict the movement of the surgical team during surgery as they try to avoid accidentally pulling or tripping on the hoses and wires and also increases the likelihood that an important instrument will be pulled onto the floor, causing damage and interruption to the surgical procedure. Thus, According to various aspects, tubes, hoses, wires, etc., including those for the surgical scope cleaner, are combined into a single tube set, which reduces the clutter in and around the sterile field. A tube set that includes a scope cleaner can be disposable and single-use, or could also be reusable in order to reduce long-term costs to the user.

According to various aspects, since the control of flow of the liquid and gas for the scope cleaner can be provided by a fluid management system, the surgical scope cleaning can be controlled by other equipment in the operating room through device control. The fluid management system can be connected to a control unit that can receive commands from surgical staff in several different ways and can transmit those commands to the fluid management system. These commands originate, for example, as voice commands, button presses on an endoscopic camera head for scrolling through menus and selecting options via the operating user's, e.g. surgeon's, display (OSD), button presses by the support staff outside of the sterile field on the touchscreen of the control unit, or on a touchscreen of a remote tablet that can be used with the control unit. According to various aspects, the liquid and gas for scope cleaning can also be controlled by button presses on the touchscreen of the fluid management system itself.

Optionally, the control unit or other device can analyze video from the endoscopic camera connected to the surgical scope with scope cleaner to detect when the image becomes blurry due to scope smudging and/or scope fogging. Upon detecting scope smudging and/or fogging, the control unit can send a command to the fluid management system to initiate a cleaning sequence.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure Optionally also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

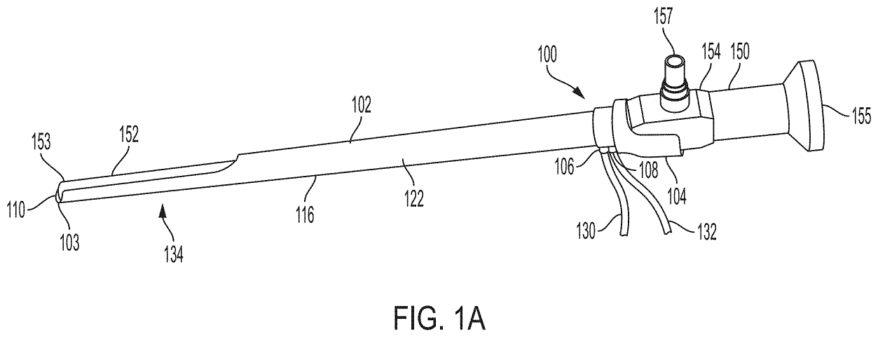

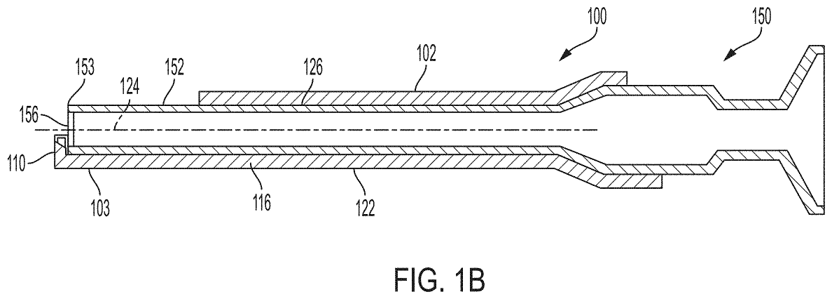

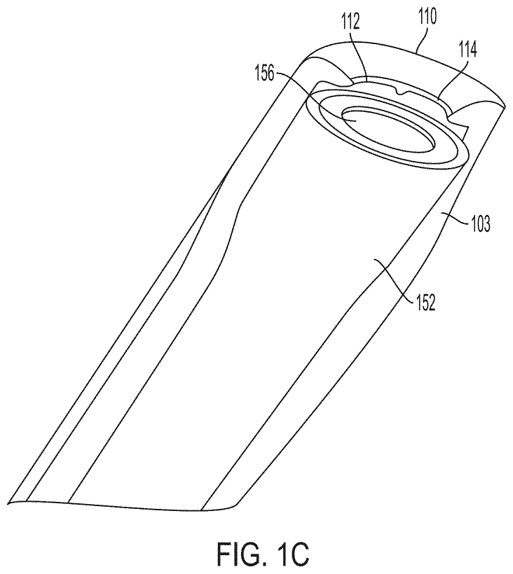

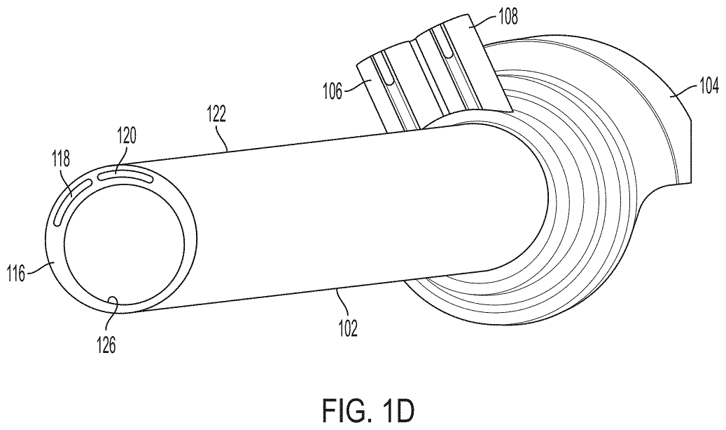

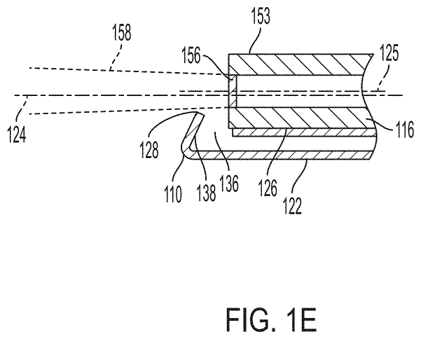

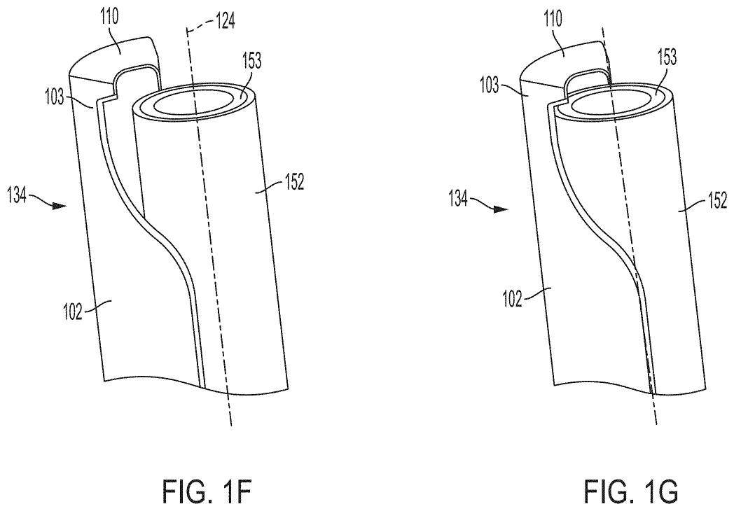

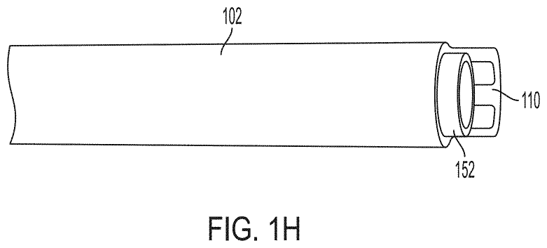

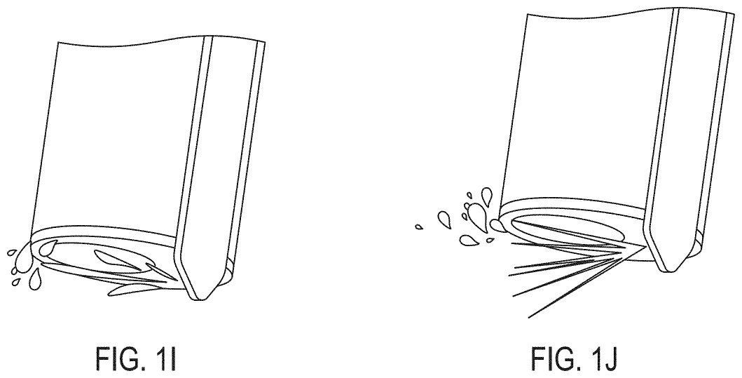

FIGS. 1A and 1B illustrate a surgical scope cleaner 100 mounted on a surgical scope 150, According to various aspects. The surgical scope cleaner 100 can be configured for mounting on a standard size surgical scope and for insertion through the cannula of a standard size trocar into a, e.g. surgical, cavity. With the surgical scope cleaner 100 mounted, the surgical scope 150 can be used for imaging, such as in the surgical cavity (along with an attached endoscope) during a surgical procedure. The surgical scope cleaner 100 can be used to remove smudging and/or condensation from the lens at the end of the surgical scope, in particular while the surgical scope remains in place in the surgical cavity during the surgical procedure. The surgical scope cleaner 100 can be used on a surgical scope that is pre-inserted into surgical cavity. The cleaning of the surgical scope per se may exclude a treatment of the human or animal body by surgery.

In the illustrated example, the surgical scope 150 includes an elongated and generally hollow tube 152 with a distal end 153 that is insertable into a body cavity, such as through the lumen of a trocar. The tube 152 extends from a housing 154 to which an eyepiece 155 is fitted to provide a viewing port through which the user, e.g. the surgeon, views the surgical field (for example, directly or through a connection between a viewing port, an endoscopic camera, and a display screen). A light port 157 extends from the housing 154 for connecting the scope 150 to an illuminator via a light cable to transmit light to a target via the scope 150. The surgical scope 150 can be, for example, a laparoscope. The surgical scope can be any type of surgical scope, including, for example, a surgical scope with an integrated camera.

The surgical scope cleaner 100 includes a sheath 102 that slides over the tube 152 of the surgical scope 150. The sheath 102 may define a generally cylindrical bore 126 that may be configured to fit to a tube of a standard size scope. The bore 126 may be sized so that the tube 152 can slide in and out of the sheath 102 while remaining radially fixed in position relative to the sheath 102 such the tube 152 and the bore 126 share substantially the same longitudinal axis 124.

A nozzle head 110 is located at a distal end 103 of the sheath 102 and extends past the distal end 153 of the tube 152 of the surgical scope 150. As explained further below, liquid and gas can be sprayed/blown from the nozzle head 110 to clean the lens at the end of the tube 152. The scope cleaner 100 is configured to remain mounted on the surgical scope as the surgical scope is being used, such as throughout a surgical procedure. The lens can be cleaned as needed without the surgical scope needing to be removed from the surgical cavity.

The sheath 102 extends from a receiver 104 that is configured to receive a housing 154 of the surgical scope 150. The receiver 104 may include one or more retention features (not shown) for retaining the housing 154 of the surgical scope 150. Optionally, retention features may orient the scope with respect to the sheath 102, which may be important for the angular scopes (scopes having an angled distal end and a lens that points at an angle from the central axis of the scope, such as 30 or 45 degrees from the axis) in order to ensure that the nozzles are directed correctly at the angled lenses. Optionally, the receiver 104 may be shaped to receive the housing 154 in the correct angular orientation for ensuring that the nozzle head 110 is properly oriented with respect to an angled scope.

A liquid port 106 and a gas port 108 are provided in the receiver 104 and may be connected to a liquid supply line 130 and a gas supply line 132, respectively, that are connected to liquid and gas supplies. Optionally, the liquid and gas ports 106, 108 extend from the sheath 102. As described further below, liquid and gas supplied through the respective ports flows through at least one conduit in the sheath to at least one nozzle in the distal end 103 of the sheath 102 for spraying liquid and blowing gas onto the lens at the distal end 153 of the tube 152 of the surgical scope 150 to clean the lens of smudges and/or condensation.

FIG. 1C illustrates the distal end 103 of the sheath 102 with the distal end 153 of the tube 152 of the surgical scope 150 received therein. The distal end 103 has a nozzle head 110 that extends past the distal end 153 of the surgical scope 150. In the illustrated example, the nozzle head 110 includes two nozzles located side-by-side—liquid nozzle 112 and gas nozzle 114. Alternatively, the nozzles are spaced longitudinally, rather than circumferentially. The nozzles are configured to direct their respective flows onto the lens 156 at the distal end of the tube 152 of the surgical scope 150. Optionally, a single nozzle is provided and both the liquid and gas conduits feed into the single nozzle.

FIG. 1D is a cross-section of the sheath 102 that illustrates the liquid conduit 118 and gas conduit 120 that extend longitudinally through the sheath 102 of the cleaner 100, According to various aspects, to provide flow paths from the liquid and gas ports 106, 108 to the liquid and gas nozzles, 112, 114 at the distal end 103 of the sheath 102. The sheath 102 includes a wall 116, and the conduits 118, 120 are formed in the wall 116 such that the conduits are completely enclosed around their longitudinal perimeter. In the illustrated example, the conduits 118, 120 are non-circular in cross-section and curve in a circumferential direction about the longitudinal axis 124 through a portion of the circumference of the wall. Curved conduits enable the outer diameter of the wall to be minimized while still providing a sufficient flow rate through the conduit. Alternatively, the conduits may be circular or any other suitable shape depending on the wall thickness and the desired flow rate and pressure drop through the conduits.

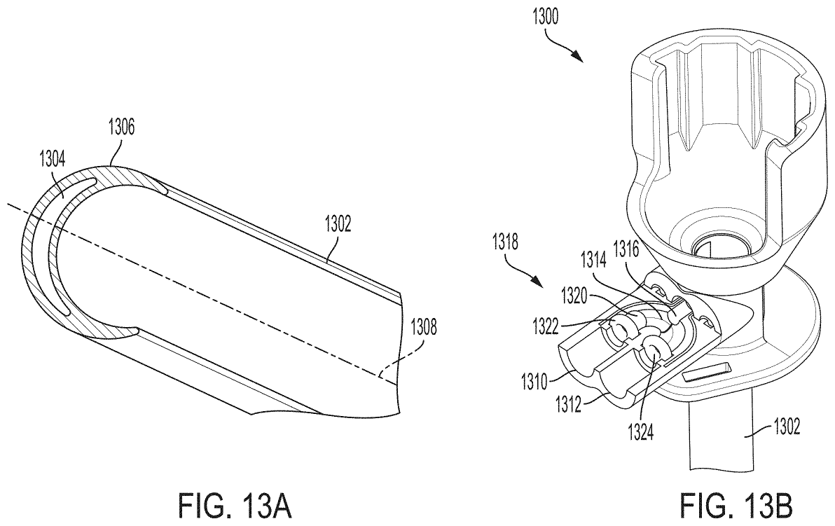

The conduits 118, 120 can extend longitudinally through the wall from the liquid and gas ports 106, 108 to the nozzle head 110. Optionally, the liquid and gas conduits merge prior to reaching the nozzle head such that a single conduit extends from a merging of the two conduits to the nozzle head 110. Optionally, the liquid and gas flow paths merge at or near the liquid and gas ports 106, 108 such that a single conduit extends substantially the entire longitudinal extent of the sheath 102. An example of this arrangement is illustrated in FIGS. 13A and 13B, and which is described in more detail below.

Returning to FIG. 1D, the bore 126 of the sheath 102 may be cylindrical and may be configured to fit to a standard tube of a surgical scope. The outer surface 122 of the sheath 102 may also be cylindrical. Optionally, the outer surface 122 of the sheath 102 may extend about a longitudinal axis 125 that is different than the longitudinal axis 124 of the bore 126 of the sheath 102 (see FIG. 1E). This off-center arrangement results in one side of the wall 116 being thicker than the other size of the wall 116, with the thicker portion of the wall accommodating the conduits 118, 120. This is shown in FIG. 1D. Thus, the wall can accommodate the conduits while keeping the outer diameter of the sheath 102 to a minimum so that the sheath 102 can fit within a standard size trocar while being mounted on a standard size surgical scope.

FIG. 1E is a cross-section of distal portions of the scope cleaner 100 and surgical scope 150. The distal end 153 of the tube 152 of the surgical scope 150 includes a lens 156. Each nozzle 112, 114 may be include a channel 136 in the nozzle head 110. A distal wall 138 of the channel 136 may be angled so that fluid moving longitudinally from the conduits 118, 120 is directed by the wall 138 toward the lens of the scope.

According to various aspects, the nozzle head 110 is configured so that the field of view of the scope 150 is not obstructed by the scope cleaner 100. The field of view of the scope 150 is represented by dashed lines 158 in FIG. 1E. The nozzle head 110 may extend radially inward of the outer diameter 160 of the tube 152, but the radially innermost portion 128 of the nozzle head 110 may be positioned with respect to the longitudinal axis 124 of the tube 152 so that the innermost portion 128 does encroach into the field of view.

Optionally, the nozzle head 110 may be configured to reduce the amount of light that may be reflected onto the lens 156. For example, at least the portion of the nozzle head 110 that faces the lens 156 may be made from a light absorbing material and/or may be colored to absorb light (e.g., colored black).

FIGS. 1F and 1G illustrate an alternative arrangement for locating the nozzle head 110 outside of the field of view of the surgical scope. In the illustrated example, the distal portion 134 of the sheath 102 is angled away from the longitudinal axis 124 of the bore 126 of the sheath 102 so that the distal end 103 of the sheath 102 is spaced away from the distal end 153 of the tube 152 in the radial direction when the cleaner 100 is mounted to the scope 150. As a result, the nozzle head 110 is well outside of the field of view of the scope 150. The sheath 102 is configured so that the distal portion 134 can be bent back against the tube 152 so that the distal portion 134 can fit within a trocar when inserting the cleaner 100 into the surgical cavity, as shown in FIG. 1G. Once the distal portion 134 is through the trocar and into the surgical field, the distal portion springs back outward. Optionally, the compliance of the material that forms the sheath 102 enables the sheath to be elastically deformed inwardly toward the tube 152 and to then spring back outward to a repeatable position.

As shown in FIG. 1A, the distal portion 134 of the sheath 102 extends only partially around the tube 152 so that the distal portion of the sheath 102 can move inward and outward relative to the tube 152. Optionally, the proximal portion of the sheath 102 can extend fully around the tube 152 while the distal portion extends only partially around the tube 152. This enables the distal portion of the sheath 102 to angle away from the tube 152 while still allowing the sheath 102 to be securely mounted on the tube 152. Optionally, the sheath 102 extends only partially around the circumference of the tube 152 along the entire length of the tube 152. According to various aspects, the length of the distal portion of the sheath 102 that extends only partially around the tube 152 is selected such that only the distal portion of the sheath—the portion that extends only partially around the tube 152—is located in a trocar during use. By having the portion of the sheath that is positioned in the trocar during use extend only partially around the tube 152, the overall size of the sheath with inserted tube 152 can be less such that a smaller trocar can be used as compared to a sheath that extends fully around the tube 152.

In variations in which the sheath 102 is configured to angle away from the tube 152, the nozzle head 110 may be made larger relative to variations in which the sheath 102 remains adjacent to the tube 152 along its entire length, which can increase manufacturability of the nozzle head 110 and/or increase nozzle performance.

FIG. 1H illustrates a variation in which the sheath is not configured to angle away from the tube 152. In this variation, the sheath 102 can extend fully around the tube 152 the full length of the sheath 102. Variations in which the sheath 102 remains adjacent to the tube 152 along the full length of the sheath can also include a sheath that does not extend fully around the tube 152 for at least a portion of the length of the sheath.

Optionally, the scope cleaner 100 is made to be disposable and can be discarded after being used in a surgical procedure. Alternatively, the scope cleaner may be configured for reuse and, as such, may be sterilizable. The scope cleaner 100 can be made of any suitable material, including any suitable plastic or metal. Examples of suitable plastics include Polycarbonate, Acrylic, Polyethylene terephthalate, Cyclic olefin copolymer, and Fluorinated Ethylene Propylene. Optionally, the scope cleaner is made via extrusion of one or more of these plastics or another suitable plastic. Optionally, the sheath 102 can be extruded out of a first plastic and the nozzle head 110 can be molded out of a different plastic and the two pieces bonded together. This might be desirable in variations in which the sheath 102 (in addition to the receiver 104 Optionally) has a first color and the nozzle head 110 has a second color, allowing for more material options for the extrusion and reduced costs and easier manufacturing. The scope cleaner can be molded, machined, 3D printed, or any combination thereof. The scope cleaner can be made of multiple components that are assembled together. For example, the nozzle head 110 may be affixed to the distal end 103 of a separate sheath 102 which may be attached to the receiver 104.

According to various aspects, the surgical scope cleaner 100 can be connected to a liquid and gas supply system that controls delivery of liquid and gas to the surgical scope cleaner during use. As such, the surgical scope cleaner 100 can be free of any valving, which can provide greater simplicity and cheaper manufacturing, which may be especially advantageous for disposable scope cleaners. Alternatively, the surgical scope cleaner can include one or more valves that may control flow of the liquid and/or gas. For example, the scope cleaner may include one or more push-button actuated valves that a user can actuate to provide the liquid spray and/or the burst of gas. One or more valves may be positioned, for example, in the receiver downstream of the ports 106, 108 or may be positioned upstream of the ports, such as in a tube set connecting the cleaner to the liquid and gas supplies.

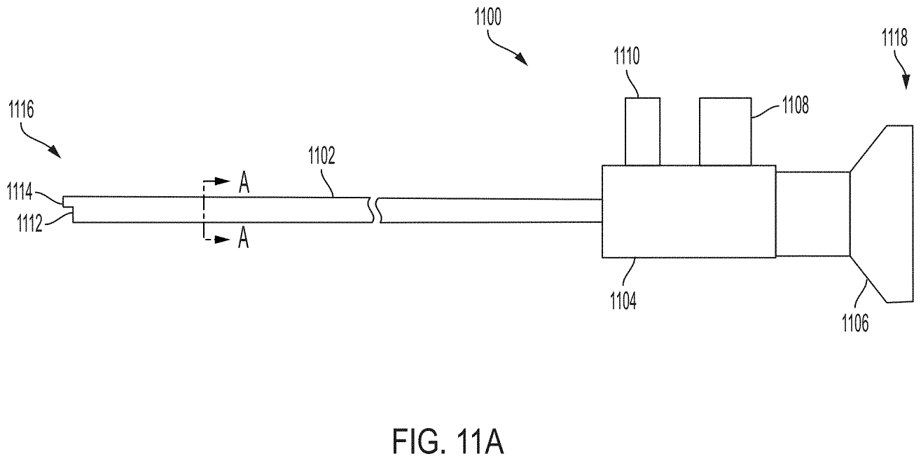

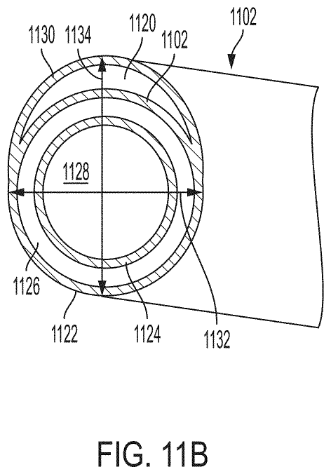

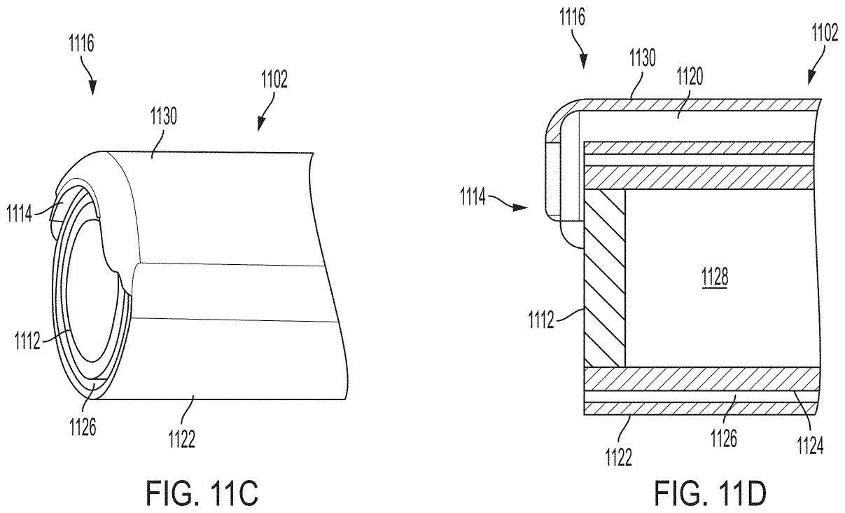

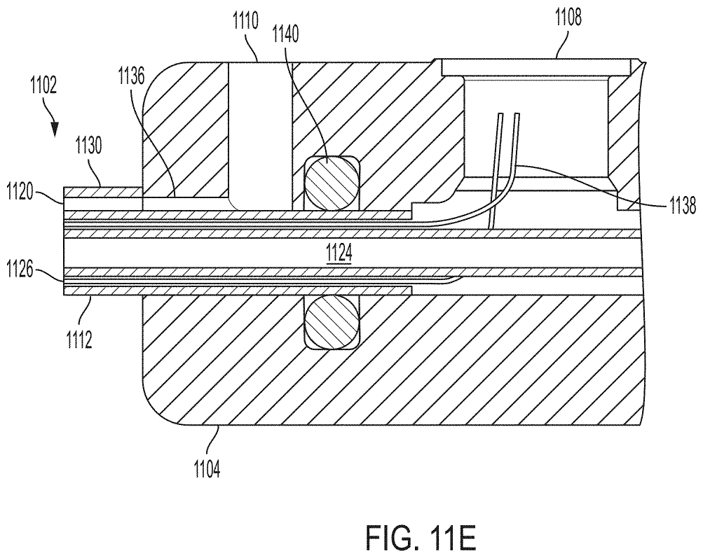

FIGS. 11 and 1J illustrate the operation of the surgical scope cleaner 100 According to various aspects. First, as shown in FIG. 1H, liquid, such as saline, is sprayed from the nozzle head 110 onto the lens 156 to remove deposits on the lens. The deposits can be, for example, blood, fat, pieces of tissue, or bile from the surgical cavity, lubricant from the seals of the trocar through which the surgical scope was inserted, fluids sprayed in the surgical cavity during the surgical procedure, such as saline for flushing or therapeutic agents, or any other substance that may deposit on the lens. The liquid may be provided to the scope cleaner 100 from a pressurized source so that the liquid impacts the lens at a velocity that is sufficient to mechanically remove the deposits. The liquid may also serve to dissolve at least some of the deposits to aid in removal. Optionally, the liquid pressure at the pressurized source is at least 1 psi, at least 3 psi, at least 5 psi, or at least 10 psi. Optionally, the liquid pressure at the pressurized source is 50 psi or less, 30 psi or less, 20 psi or less, or 10 psi or less.

Next, as shown in FIG. 1J, a jet of gas is delivered from the nozzle head 110 onto the lens to remove the liquid and any loosened deposits remaining on the lens. The gas may be, for example, carbon dioxide, which is commonly available in the surgical field such as for insufflating the surgical cavity. As with the liquid, the gas may be provided from a pressurized gas source so that a burst of the gas is blown onto the nozzle with a relatively high velocity. Optionally, the gas pressure at the pressurized gas source is at least 5 psi, at least 10 psi, at least 50 psi, or at least 100 psi. Optionally, the gas pressure at the pressurized gas source is 500 psi or less, 250 psi or less, 150 psi or less, or 100 psi or less. The burst of the gas blows the sprayed liquid and any remaining deposits off of the lens, leaving the lens clean and clear. To the extent that some deposits remain, the cleaning sequence can be repeated as necessary.

Optionally, at least a portion of the period that the gas is delivered can overlap with at least a portion of the period of liquid spray. This can increase the velocity of the liquid spray, increasing its cleaning power.

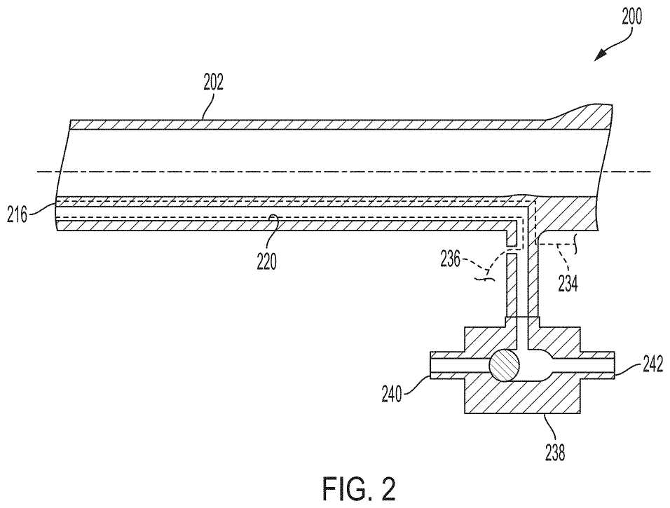

Optionally, the scope cleaner may be configured for preventing fogging of the lens of the scope by warming the surgical scope and/or providing a steady stream of gas to the surgical scope. FIG. 2 is a cross section of a portion of a scope cleaner 200 that is configured for warming and for providing a steady stream of gas to a surgical scope, According to various aspects. One or more resistive heating wires 234 may be incorporated into the sheath 202 for warming to prevent fogging. One or more wires 234 can be molded into the wall 216 of the sheath 202 to warm the tube of a surgical scope received therein. Alternatively or additionally, one or more wires 236 can extend within the gas conduit 220 to warm the gas as it flows through. The one or more wires 234 and/or 236 can be connected to an electrical source via wiring that, for example, is incorporated into a tube set that includes tubes carrying liquid and gas for the scope cleaner 100.

Optionally, the scope cleaner 200 is configured for providing a steady stream of gas for preventing fogging while also providing a burst of gas for the cleaning sequence. The cleaner 200 may include a shuttle valve 238 that has two separate gas inlets 240 and 242 for connecting to two separate gas lines. A first gas inlet 240 can be used for providing low pressure gas that, when flowing, provides a steady stream of gas down the gas conduit 220 and out onto the lens of the scope. The low pressure gas could be regulated to be, for example, 2 psi or less. A burst of high pressure gas through the second gas inlet 242 will force the shuttle valve 244 to the position closing off the low pressure line, opening the flow path for the high pressure burst, which will flow down the gas conduit 220 to the lens of the scope. When the high pressure burst is finished, the pressure from the low pressure gas will shuttle the shuttle valve back to the position allowing the low pressure gas to flow.

Optionally, the low pressure gas flow can be the insufflating gas flow for insufflating the surgical cavity, which can eliminate the need for a separate insufflating line and insufflating inlet to the surgical cavity. Optionally, a scope cleaner is configured for scope heating, gas heating, and/or steady gas flow.

According to various aspects, the liquid and gas supplies for the surgical scope cleaner can be incorporated into an apparatus that manages the flow of other fluids into and out of the surgical field. In addition to the liquid and gas supplies for the surgical scope cleaner, examples of other fluid management that can be provided, according to various aspects, include providing insufflating gas for pressurizing the surgical cavity of a patient, evacuating smoke that may be created in the surgical cavity via cauterization, supplying irrigation liquid within the surgical cavity, removing liquid from the surgical cavity, and supplying of therapeutic agents to the surgical cavity.

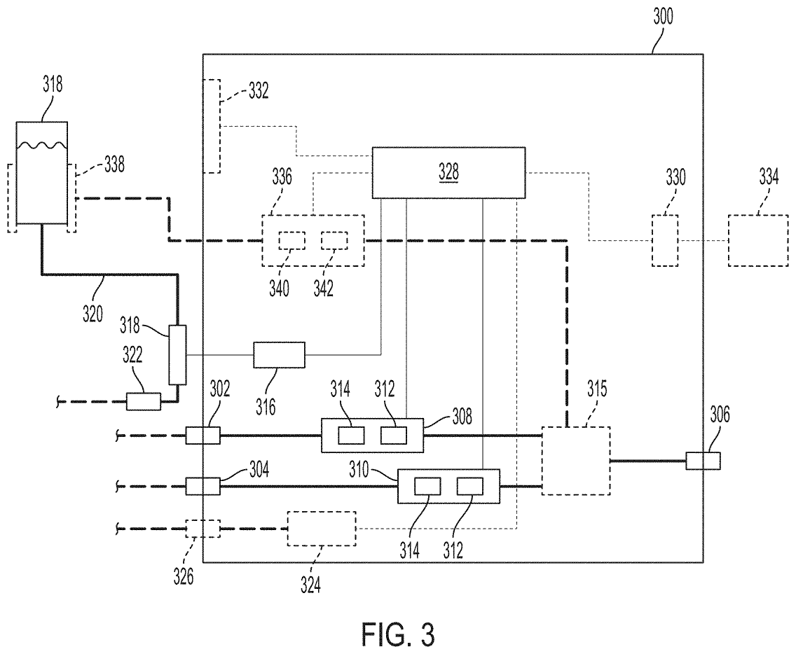

FIG. 3 is a block diagram of an apparatus 300 for managing fluid flow into and out of a surgical field, According to various aspects. Apparatus 300 controls the flow of liquid and gas to a surgical scope cleaner, such as scope cleaner 100, and the flow of insufflating gas for pressurizing a surgical cavity. Apparatus 300 can be configured for managing the flow of any other fluids that are needed for the surgical field. A surgical scope cleaner and a device, such as a trocar, for directing insufflating gas into the surgical cavity can be connected to the apparatus 300 via, for example, flexible tubing that extends from the apparatus 300 into the surgical field.

Apparatus 300 includes a first gas supply port 302 for providing the gas supply to the surgical scope cleaner and a second gas supply port 304 for supplying an insufflating gas flow to the surgical cavity of a patient. The apparatus 300 includes a gas inlet port 306 for supplying gas to the apparatus 300. The gas inlet port 306 can be connected to a pressurized gas supply, such as a carbon dioxide wall or service head outlet or a carbon dioxide canister.

Gas supplies to the first and second gas supply ports 302, 304 can be controlled via first and second gas flow control subsystems 308 and 310, respectively. Each gas flow control subsystem can include, for example, a pressure regulator 312 for stepping down the pressure of the gas supplied to the apparatus 300 and a valve 314 for turning the flow of gas to the respective port 302, 304 on and off. Optionally, a single pressure regulator 315 may be used for both the first and second gas supply ports 302, 304.

Apparatus 300 also includes an actuator 316 for controlling flow of liquid to the surgical scope cleaner. The actuator 316 may be operatively connected to a flow device 318 that connects a liquid supply reservoir 320 to a liquid supply port 322. The surgical scope cleaner can be connected to the liquid supply port 322 via tubing for receiving liquid from the liquid supply reservoir 320 as controlled by the actuator 316 and flow device 318.

The actuator 316 and flow device 318 can be implemented in different ways according to various aspects. Optionally, the flow device 318 is a valve that is moved between open and closed positions by the actuator 316. Alternatively, the flow device 318 is a flexible tube that is compressed by the actuator 316 to close of the flow path through the flow device 318. The actuator 316 can be a linear actuator, such as a solenoid, that operates the valve or compresses the flexible tube. Alternatively, the actuator 316 is a rotary actuator, such as a stepper motor or servomotor, that rotates the valve between open and closed positions. Optionally, the flow device 318 is a pump that is actuated by the actuator 316. The actuator 316 may be, for example, a motor that rotates a shaft onto which the pump is mounted.

In the illustrated example, the flow device 318 is separate from and external to the apparatus 300, which results in the liquid flow path being entirely external to the apparatus 300. This arrangement can be advantageous in that there are no apparatus components that need sterilization. Alternatively, the flow device 318 may be included in or otherwise as part of the apparatus 300.

The liquid supply reservoir 320 can be any suitable reservoir for providing the liquid needed for scope cleaning. For example, the liquid supply reservoir 320 can be a saline bag that is connected to the flow device 318 via tubing or can be combined with the flow device 318 into a single unit. Optionally, the liquid supply reservoir 320 is incorporated into the apparatus 300.

The apparatus 300 can include other fluid supply or discharge components. For example, the apparatus 300 can include a vacuum controller 324 for providing vacuum to the surgical field via a vacuum port 326. The vacuum can be used, for example, for evacuating smoke from the surgical field and/or suctioning liquid, such as blood, from the surgical field.

Optionally, the apparatus 300 includes a liquid reservoir pressurization subsystem 336 for pressurizing the liquid supply reservoir 320 using the same gas as used for the scope cleaning or a different gas. The pressurization subsystem 336 can provide pressurized gas, such as gas from the gas inlet port 306, to the liquid supply reservoir 320. Optionally, the pressurized gas can create head pressure in the reservoir 320. Alternatively, the pressurized gas can compress the reservoir itself. For example, the reservoir may be a saline bag fitted within a pressurization sleeve 338 that receives the pressurized gas from the liquid reservoir pressurization subsystem 336.

The liquid reservoir pressurization subsystem 336 can include one or more valves 340 for controlling flow of pressurized gas, which can be the same gas as provided via the gas inlet port 306. Optionally, the liquid reservoir pressurization subsystem 336 can include a pressure regulator 342 for stepping down the pressure received via the inlet port 306 or via one or more upstream regulators, such as regulator 315. Optionally, the apparatus can be configured to control the liquid reservoir pressurization subsystem 336 to automatically depressurize the pressurization sleeve 338 at the end of a surgical procedure, such as when the insufflation is stopped.

Optionally, the apparatus 300 can be configured to provide at least three gas pressures. The lowest pressure gas delivery subsystem could be used for safe insufflation of the patient, a higher pressure gas delivery subsystem could be used to pressurize the liquid reservoir for use in irrigation during the surgery, and a highest pressure gas delivery subsystem could be used for things that require high-pressure bursts of gas, such as blow-drying the laparoscope after it has been washed. This high-pressure gas can be preferred for other tools or accessories that can be attached to the tube set and driven by the fluid management apparatus, such as a sprayer for therapeutic agents or even a gas-driven tool or instrument that can be used to do work during the surgical procedure (such as a rotary tool or sagittal tool).

The gas flow control subsystems 308, 310, the actuator 316, and any other electronic component of the apparatus 300 can be control via a controller 328. The controller 328 may include one or more processors and memory that stores instructions for execution by the one or more processors for controlling fluid management by the apparatus 300. The controller 328 may provide electrical signals to one or more valves and/or pressure regulators of the control subsystems 308, 310 and to actuator 316 actuating the actuator 316. The controller can also be used to control any other fluid management subsystems, including the vacuum controller 324 and the liquid reservoir pressurization subsystem 336.

The controller 328 may be communicatively connected to an external control system 334 via a communication port 330 for receiving liquid and gas supply control commands from the external system 334. For example, the controller 328 may receive a command to execute a cleaning sequence for the surgical scope cleaner, and in response, the controller may control the actuator 316 for supplying the liquid to the surgical scope cleaner for a predetermined period of time for spraying onto the lens of the surgical scope, as discussed above, and control the first gas flow control subsystem 308 for supplying a gas flow to the surgical scope cleaner for a second predetermined period of time for blowing liquid off of the lens of the surgical scope.

The apparatus 300 may include a user interface 332 for a user to control one or more aspects of the liquid and gas supply from the apparatus. The user interface 332 may be used, for example, for receiving commands for starting and stopping the insufflating gas flow and/or changing the insufflating gas pressure or for controlling any other function of the apparatus 300, according to various aspects.

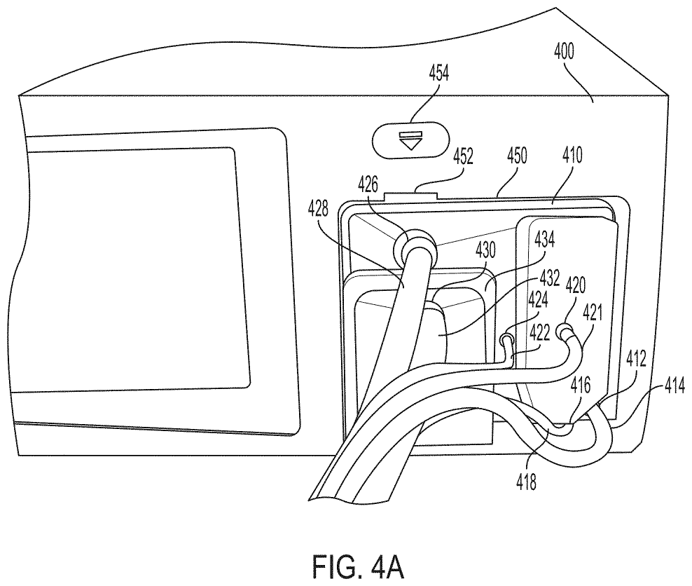

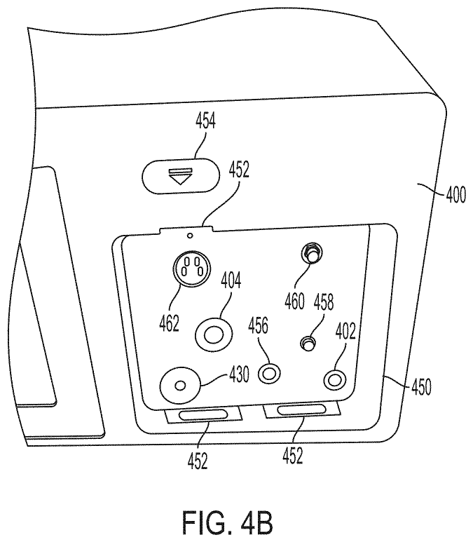

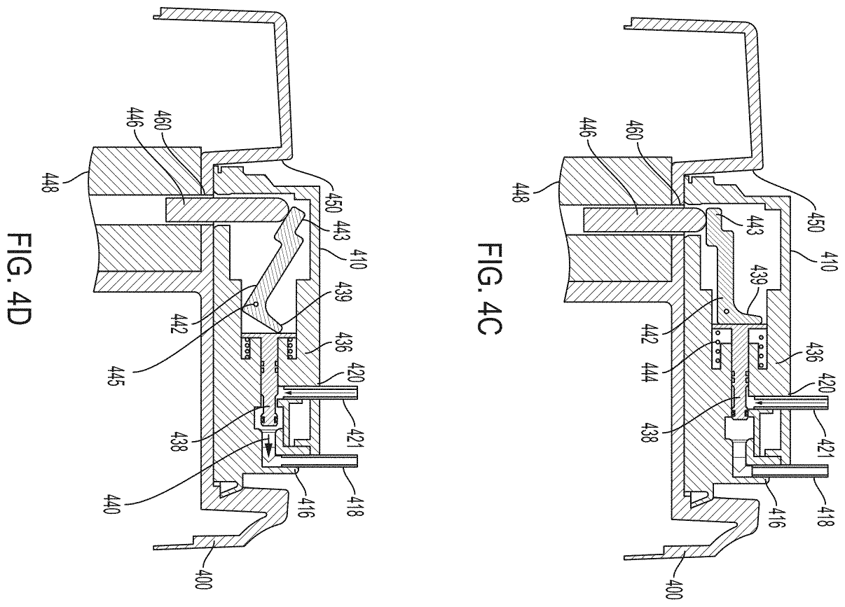

Optionally, lines for conducting fluids managed by a fluid supply apparatus, such as apparatus 300, to the surgical field are connected directly to the ports of the apparatus. Alternatively, the supply lines are connected to a connector that is connected to a receptacle of the apparatus. FIG. 4A illustrates a fluid supply apparatus 400 in which a connector 410 is used to connect multiple fluid lines to the apparatus 400.

The connector 410 includes a gas supply port 412 for supplying gas to a surgical scope cleaner via a gas supply line 414, a liquid supply port 416 for supplying liquid to the surgical scope cleaner via a liquid supply line 418, and a liquid inlet port 420 for receiving liquid from an external liquid reservoir for the surgical scope cleaner via a liquid inlet line 421. A liquid reservoir pressurization port 424 can be included for supplying pressurized gas to a liquid supply reservoir, such as reservoir 320 of FIG. 3, via a liquid reservoir pressurization line 422.

The connector 410 also includes an insufflating gas supply port 426 for supplying an insufflating gas flow to the surgical cavity via an insufflating gas line 428 and a smoke evacuation port 430 for evacuating smoke from the surgical cavity via a smoke evacuation line 432. The connector 410 can include an inflow filter housing 434 that houses one or more filters for filtering smoke received via the evacuation port 430. The connector 410 can include other filters for filtering fluid provided to and received from the surgical field.

The connector 410 may be removably received in a receptacle 450 of the apparatus 300. One or more latches 452 may be used to retain the connector 410 in the receptacle 450. One or more ejection mechanisms 454 can be used to release the one or more latches 452 for removing the connector 410 from the receptacle 450.

FIG. 4B illustrates the receptacle 450, According to various aspects. The receptacle 450 includes the first gas supply port 402 for supplying gas flow to the surgical scope cleaner, a second gas supply port 404 for supplying insufflating gas flow to the surgical cavity, and a smoke evacuation port 430 for evacuating smoke from the surgical cavity. The receptacle 450 also includes a liquid reservoir pressurization port 456 for providing pressurization gas to a liquid supply reservoir, such as liquid supply reservoir 320 of FIG. 3.

The receptacle 450 may include a switch 458 that is depressed or otherwise actuated when the connector 410 is received in the receptacle 450. The switch 458 may be connected to a controller, such as controller 328 of FIG. 3, which may control the flow of one or more fluids based on the status of the switch so that there is no flow through one or more of the ports of the receptacle 450 when the connector 410 is not received in the receptacle 450. The receptacle 450 also includes an aperture 460 through which an end of an actuator for actuating a flow control device in the connector 410 extends, as discussed further below. The receptacle 450 may also include an electrical connection 462 for providing electricity to one or more heated tubes connected to the connector 410.

The rear side (not shown) of the connector 410 includes ports that fit to the ports of the receptacle 450 described above. One or more seals may be provided on any of the ports of the receptacle 450 and/or on any of the ports of the rear side of the connector 410. The rear side also includes an aperture for receiving the end of the actuator.

FIGS. 4C and 4D are cross sections of the connector 410 located in the receptacle 450, illustrating a flow device 436 incorporated into the connector 410. Flow device 436 includes a valve 438 that is moved laterally to open and close a liquid flow path 440 through the flow device 436. FIG. 4C illustrates the closed position and FIG. 4D illustrates the open position of the valve 438. The valve 438 is moved from the closed position to the open position by one end 439 of a lever 442 and is returned to the open position through the force of a spring 444. The lever 442 pivots about a pivot axis 445. To actuate the valve 438, a plunger 446 of a solenoid actuator 448 in the apparatus 400 pushes on the other end 443 of the lever 442, causing the lever to pivot about the pivot axis 445, pushing the end 439 of the lever 442 against the valve 438, forcing the valve to move laterally to the open position against the force of the spring 444. As shown in FIG. 4D, with the valve 438 in its open position liquid from a liquid supply reservoir can flow through the flow device 436 via the liquid inlet port 420 and out to the surgical scope cleaner via the liquid supply port 416.

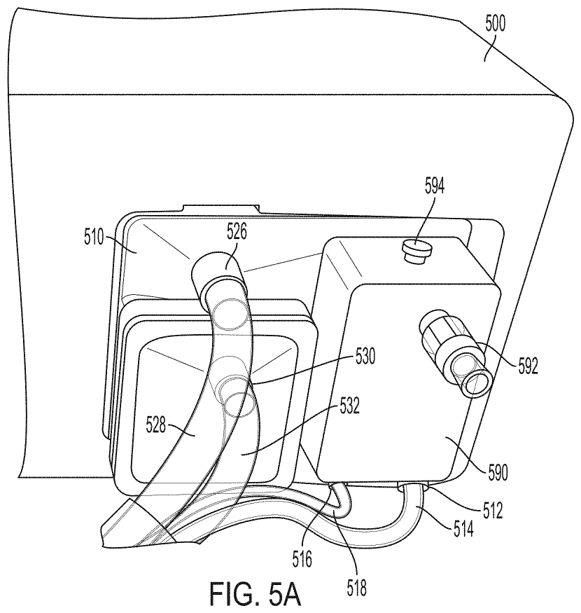

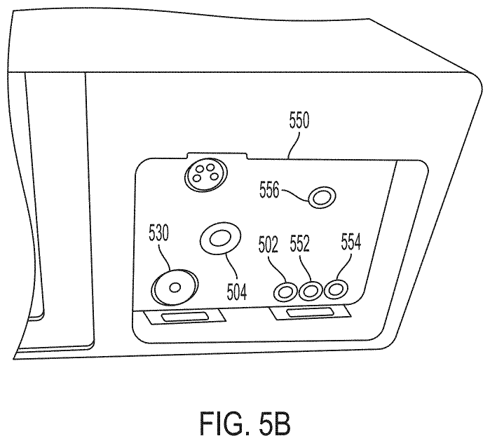

FIGS. 5A-5B illustrate a fluid supply apparatus 500 and tube set connector 510 in which a liquid supply reservoir for supplying the liquid for the scope cleaner is incorporated into the connector 510, According to various aspects. Similarly to connector 410, connector 510, as shown in FIG. 5A, includes a gas supply port 512 for supplying gas to a surgical scope cleaner via a gas supply line 514, a liquid supply port 516 for supplying liquid to the surgical scope cleaner via a liquid supply line 518, an insufflating gas supply port 526 for supplying an insufflating gas flow to the surgical cavity via an insufflating gas line 528, and a smoke evacuation port 530 for evacuating smoke from the surgical cavity via a smoke evacuation line 532. However, unlike connector 410, connector 510 does not have a liquid supply port for receiving liquid from an external liquid reservoir. Instead, connector 510 includes a liquid reservoir 590 built in.

The connector 510 can include a reservoir filling port 592 for filling the liquid reservoir 590 with liquid, such as saline. The filling port 592 may have a one-way valve for sealing the port when the reservoir 590 is pressurized, as discussed further below. A bleeder valve 594 may be provided for bleeding air when filling the reservoir 590.

FIG. 5B illustrates the receptacle 550 of the apparatus 500 that receives the connector 510, According to various aspects. The receptacle 550 includes the first gas supply port 502 for supplying gas flow to the surgical scope cleaner, a second gas supply port 504 for supplying insufflating gas flow to the surgical cavity, and a smoke evacuation port 530 for evacuating smoke from the surgical cavity. The receptacle 550 also includes a liquid reservoir pressurization port 556 for providing pressurized gas to the liquid reservoir 590 to pressurize the liquid provided to the scope cleaner.

According to various aspects, a flow device in the form of a valve is provided within the apparatus 500 for controlling the flow of liquid from the reservoir 590. Accordingly, the receptacle 550 includes a liquid inlet 552 that receives liquid from the reservoir 590 (via a connection with an outlet on the back of the connector 510, which is not shown) and a liquid outlet 554 for providing the liquid to the scope cleaner via the connector 510. The flow device is provided in a flow line that extends between the liquid inlet 552 and outlet 554. The flow device is actuated by an actuator, such as a solenoid, so that the liquid flow can be turned on and off. Once the reservoir 590 is pressurized, opening the valve of the flow device allows liquid to flow to the scope cleaner.

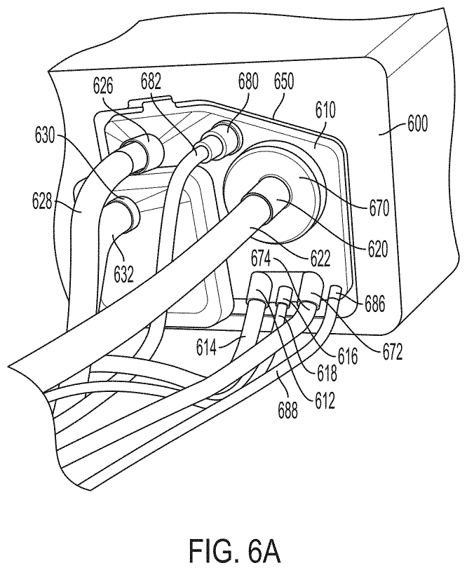

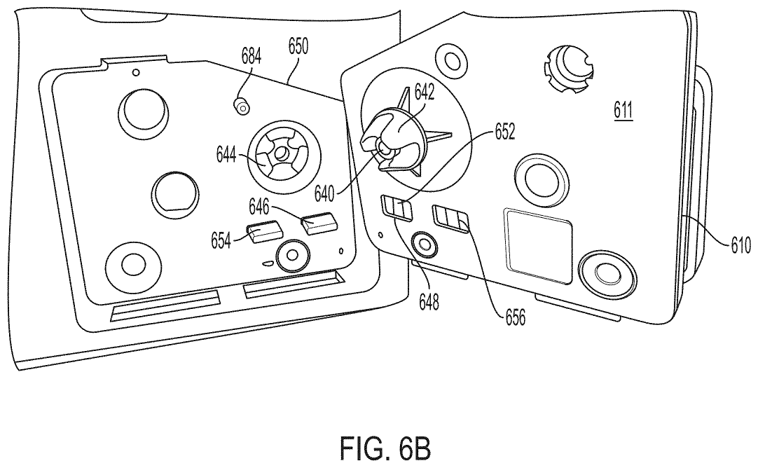

FIGS. 6A and 6B illustrate a fluid supply apparatus 600 and tube set connector 610 in which a pump is incorporated into the connector 610 for pumping liquid from an external liquid supply reservoir to the scope cleaner, According to various aspects. The connector 610 includes a gas supply port 612 for supplying gas to a surgical scope cleaner via a gas supply line 614 and a liquid supply port 616 for supplying liquid to the surgical scope cleaner via a liquid supply line 618. A liquid inlet port 620 for receiving liquid from an external liquid reservoir, such as reservoir 320 of FIG. 3, via a liquid inlet tube 622 can lead into a pump impeller housed within a pump housing 670. The connector 610 also includes an insufflating gas supply port 626 for supplying an insufflating gas flow to the surgical cavity via an insufflating gas line 628 and a smoke evacuation port 630 for evacuating smoke from the surgical cavity via a smoke evacuation line 632.

FIG. 6B shows the receptacle 650 and the rear side 611 of the connector 610 that interfaces with the receptacle 650. Extending through the rear side 611 of connector 610 is a shaft 640 for a pump impeller housed within the connector 610. Mounted to the shaft 640 is a first coupler 642 that couples to and is driven by a second coupler 644 in the receptacle 650. The second coupler 644 is mounted to a shaft of a motor located within the apparatus 600. The motor drives the pump impeller to pump the liquid from the external liquid supply reservoir to the scope cleaner.

The pump can be started and stopped to control flow of liquid to the scope cleaner. Additionally or alternatively, an actuator can be used to open and close a liquid flow path in the connector 610. In the example illustrated in FIG. 6B, a plunger 646 of the actuator, which can be in the form of a solenoid, extends from the receptacle 650 and is received in an aperture 648 in the rear side 611 of the connector 610. The plunger 646 extends to a liquid flow line 652 in the connector 610, which leads from the pump to the liquid supply port 616. The plunger 646 can be extended through action of the solenoid to pinch off the liquid flow line 652. Thus, in this example, the flow device is the liquid flow line 652, which is pinched down by the plunger 646 of the actuator to shut off flow of the liquid. The pump can run continuously and flow of liquid to the scope cleaner can be controlled by the pinching of the liquid flow line 652. Optionally, the flow from the pump could also be controlled via a valve, such as valve 438 of FIGS. 4C-4D. Optionally, pump may be continuously pressurizing the liquid and flow can be controlled by, for example, a pinching actuator or a valve.

According to various aspects, a pinching actuator can also be used for controlling flow of one or more other fluids, including, for example, the gas supply for the scope cleaner. FIG. 6B illustrates a second plunger 654 extending from the receptacle 650 for pinching a second flow line 656 in the connector 610. The second flow line can be, for example, a portion of the scope cleaner gas flow path through the connector 610. This arrangement can eliminate the need for a valve located in the apparatus for controlling the gas flow to the scope cleaner. Optionally, a valve is provided for controlling flow through second flow line 656.

According to various aspects, including a pump in the connector 610 provides the ability for the apparatus to manage supply of liquid to the surgical field for additional purposes, such as for irrigation within the surgical cavity. One or more additional liquid flow path lines can lead from the pump to one or more additional tubes extending from the connector 610. Referring back to FIG. 6A, an irrigation outflow port 672 can be included for providing an irrigation outflow from the pump, via an irrigation tube 674, to an irrigation supply device used to irrigate the surgical cavity. Optionally, the second plunger 654 or an additional plunger, can be used to pinch a flow line for the irrigation liquid in the connector 610 for controlling the flow of the irrigation fluid.

According to various aspects, a connector, such as connector 610, can include wires, cables, or other lines for providing electricity and/or data communication. In the example illustrated in FIGS. 6A-6B, the connector 610 includes a monopolar RF connector 680 and cable 682 for providing electricity to an electrocautery instrument in the surgical field. The connector 680 can interface with a power port 684 in the receptacle 650, as shown in FIG. 6B. Connector 610 can also include a control signal connector 686 for connecting a control signal wire 688 that can be used to control one or more functions of the apparatus 600, such as the delivery of liquid and/or gas, the operation of the pump, or any other function. The wire 688 can lead, for example, to a switch or other remote control located in the surgical field that can be operated by a user.

According to various aspects, an integrated tube set can be used to provide fluids managed by a fluid supply management apparatus, such as apparatus 300, 400, 500, or 600, to the surgical field. The integrated tube set can reduce the clutter in the surgical field by collecting fluid supply lines together. The integrated tube set can include a connector to which some or all of the lines are connected, such as any of connector 410, connector 510, or connector 610, which can simplify the set-up process for connecting the lines to one or more pieces of equipment. An integrated tube set can also include one or more integrated devices that can be used in the surgical field to deliver fluids to or from the surgical field, such as a surgical scope cleaner and a suction/irrigation device. Integrated tube sets can be disposable, single-use tube sets or can be reusable tube sets that are sterilized between each use.

According to various aspects, the fluid supply management apparatus can be configured to accept various configurations of tube sets, such as a tube set that has only a subset of available tubes, which can be a tube set that has just an insufflation inflow line and an insufflation outflow line, a tube set that has just insufflation lines and suction and irrigation lines, a tube set that has insufflation lines and suction and irrigation and a scope cleaner, and tube sets that have any other accessories such as a therapeutic agent sprayer or other tool or instrument driven and controlled by the fluid management apparatus. The fluid management apparatus can be configured to recognize what configuration of tube set is attached and can adjust its control to supply fluids in a way that is tailored to that particular type of tube set. The tube set recognition can be facilitated by, for example, RFID, such as by including an RFID reader in the receptacle 650 of apparatus 600 and an RFID tag in the connector 610 of the tube set. Optionally, electromechanical switching mechanisms can be included in the fluid supply management apparatus that respond to physical features on the portion (e.g., connector 610) of the tube set that is connected to the apparatus.

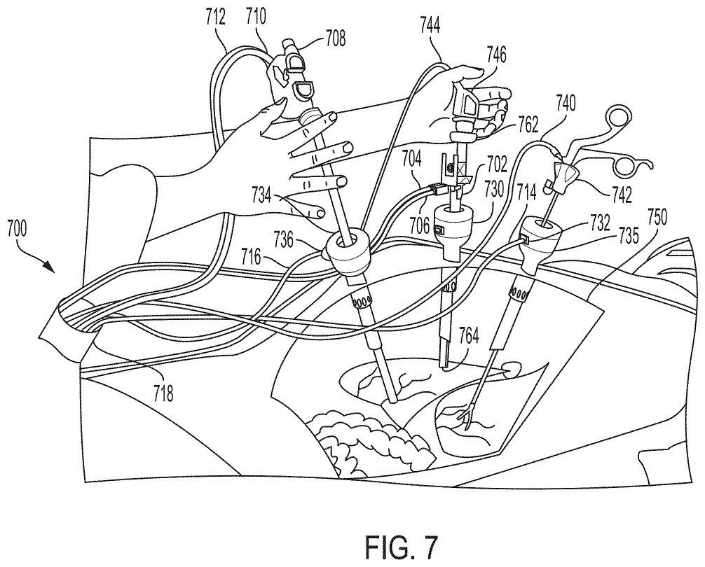

FIG. 7 illustrates the use of a tube set 700, According to various aspects, in a surgical field. The tube set 700 includes surgical scope cleaner 702 configured in accordance with the principles discussed above, which is connected to a scope cleaning gas supply tube 704 and a scope cleaning liquid supply tube 706. In this variation, the surgical scope cleaner 702 is an integrated component of the tube set in which the scope cleaner and connected supply tubes are pre-connected, but Alternatively, the scope cleaner is not provided as a part of the tube set, and the supply tubes 704 and 706 of the tube set are connected to a scope cleaner in preparation for a surgical procedure, such as in the operating room. The surgical scope cleaner 702 can be mounted to a surgical scope 762 fitted to an endoscopic camera 746. The surgical scope cleaner 702 and surgical scope 762 can be inserted into the surgical cavity 750 through a first trocar 730 for visualizing the surgical cavity. When the lens 764 at the end of the surgical scope 762 gets smudged or fogs, the scope cleaner 702 can be used to clean the lens 764 in accordance with the principles discussed above.

The tube set 700 can include a suction and irrigation device 708 that is connected to an irrigation supply tube 710 and a suction tube 712. The suction and irrigation device 708 can be inserted into a second trocar 734 for providing suction and irrigation in the surgical cavity 750. The suction and irrigation device 708 can be an integrated component of the tube set or can be connected to the tube set in preparation for a surgery.

The tube set 700 can also include an insufflation gas supply tube 714, which can be connected to a port 732 of a third trocar 735 for providing pressurized gas to the surgical cavity 750. The tube set also includes a patient outflow tube 716 that can be connected to a port 736 of the second trocar 734 for withdrawing gas, such as smoke, from the surgical cavity 750.

The tubes of the tube set 700 can be held together by an outer tube 718, which can help declutter the operating room. Optionally, the tubes are held together by one or more straps that are wrapped around the tubes. The tube set can include other lines that extend into the surgical field, such as a monopolar line 740 for providing current to a cauterization tool 742. The tube set could also include one or more data lines 744 and/or a light cable for connecting a camera control unit and/or an illuminator to an endoscopic camera 746 that is mounted to the surgical scope 762. Optionally the scope cleaning supply tubes are attached to the fiber optic light cable, which is already attached to the scope directly adjacent to where the two scope cleaner tubes connect to the scope-cleaning sheath. The tubes can be attached to the light cable with either a clip, a Velcro strap, or other similar method. The clip or strap can be permanently installed onto the either the light cable or the scope cleaner tube set by the manufacturer, or it could be a separate item that gets installed by the surgical staff at the start of the surgical procedure. Tube sets, according to various aspects, can incorporate a laparoscopic sprayer that can convey pressurized gas from the fluid management apparatus to spray therapeutic agents inside the surgical cavity, such as hemostatic agents.

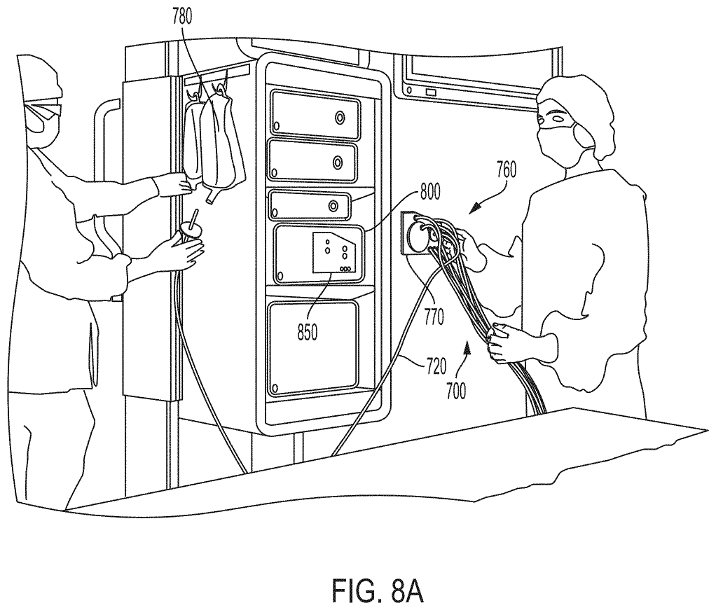

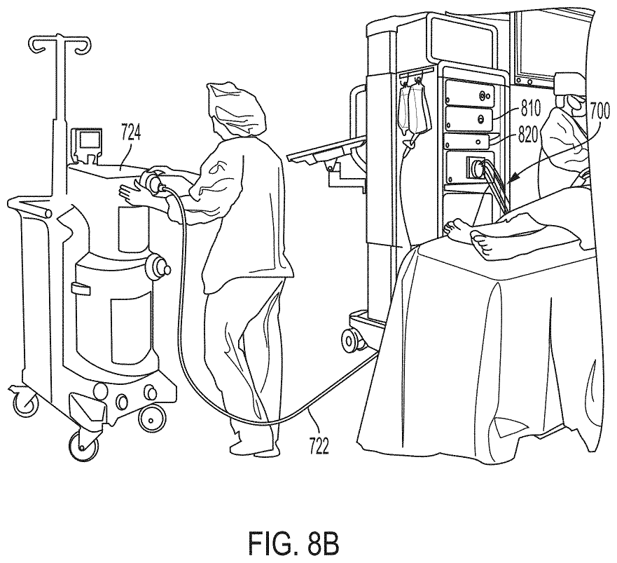

FIGS. 8A and 8B illustrate the connection of tube set 700 to a fluid supply management apparatus 800, such as any of apparatus 300, 400, 500, and 600 discussed above, According to various aspects. The non-patient end 760 of the tube set 700 includes a connector 770, such as any of connectors 410, 510, and 610, to which some or all of the tubes of the tube set are pre-attached. The connector 770 is connected to the receptacle 850 of the fluid supply management apparatus 800. In the illustrated example, the tube set 700 includes a liquid supply tube 720 that is connected to a liquid supply reservoir 780, which can be for example a saline bag, for supplying liquid to the surgical field, such as for the scope cleaner and/or irrigation supply device. FIG. 8B illustrates the connection of a suction line 722 of the tube set 700 to a suction apparatus 724.

Although not shown, one or more lines in the tube set can be connected to other equipment in the operating room. For example, one or more communication lines for an endoscopic camera can be connected to a camera control unit 810 and a light cable can be connected to an illuminator 820.

Optionally, a tube set can be configured so that the gas line connected to the scope cleaner can be disconnected from the scope cleaner and attached to another device used during the surgical procedure, such as a spraying wand to spray, for example, a hemostatic curing agent onto wound sites within the surgical cavity or medications to provide therapeautic healing effects to areas of the surgical cavity. The pressurized gas could be used to provide the power for a gas-driven instrument or power tool. Optionally, the liquid line connected to the scope cleaner can be disconnected from the scope cleaner for powering another device used in the surgical field. Optionally, both the gas and liquid lines could be disconnected from the scope cleaner and used for powering and/or controlling another device used in the surgical field. Optionally, a signal line from the fluid delivery system can be provided to connect to the device that the liquid and/or gas lines are connected to, whether the scope cleaner or any other device that interfaces with the liquid and/or gas lines. Information related to the type of device to which the line(s) are connected to the fluid delivery system can be communicated via this signal line so that the fluid delivery system can provide liquid and/or gas flows that are suitable for the connected device. For example, when the lines are connected to the scope cleaner, the fluid delivery system may register that the scope cleaner is connected (via the signal on the signal line) and may provide the liquid and gas flows per the cleaning sequence, and when the liquid and/or gas lines are connected to a device that is powered by the liquid and/or gas, the fluid delivery system may recognize this connected via the signal line and may provide the liquid and/or gas flows continuously.

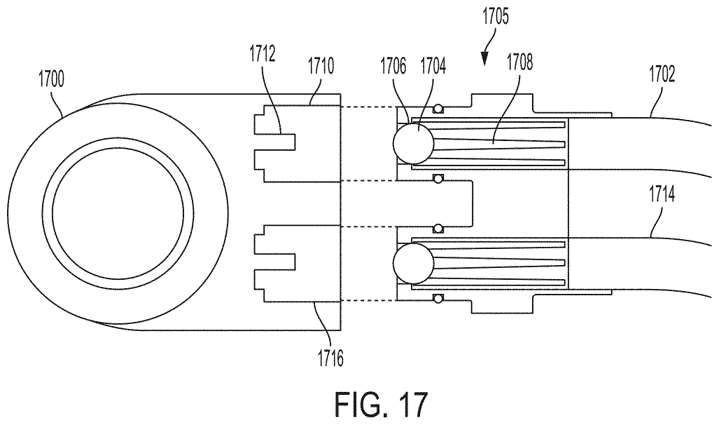

According to various aspects, one or more tubes supplying liquid and/or gas to an apparatus in the surgical field (such as a trochar, a surgical scope cleaner, an irrigator, etc.) can be configured to be detached from the device without liquid or gas continuing to flow out of the tube. This could be useful, for example, to allow for switching out devices during the procedure without having to interact with the fluid supply management apparatus. To accomplish this, the tube set can include a valve that is normally closed and that is actuated when the tube engages with the port of the device. FIG. 17 illustrates an example of such an arrangement for a surgical scope cleaner 1700. A supply tube 1702 includes a ball 1704 that seals against a seat 1706 located at the distal end 1705 of the supply tube 1702, preventing fluid flow out of the distal end. The ball 1704 can be held against the seat 1706 by fluid pressure and/or by a biasing mechanism 1708, such as a spring. The corresponding port 1710 of the scope cleaner 1700 can include an actuation member 1712 that engages with the ball 1704 when the tube 1702 is connected to the port 1710. As the tube 1702 is pushed onto the port 1710, the ball 1704 is pushed away from the seat 1706 by the actuation member 1712 (e.g., a finger-like protrusion), which enables fluid to flow around the ball 1704 and through the distal end 1705 of the tube 1702 and into the scope cleaner 1700. When the tube 1702 is removed from the port 1710, the ball 1704 reseats due to the pressure of the fluid and/or due to the force of the biasing mechanism 1708, which cuts off fluid flow. The other tube 1714 and port 1716 can have a similar valve arrangement.

This quick-connect arrangement can be applied to any other tube connection, including for a suction and irrigation device. It can be advantageous (and lower cost) for the surgical staff to stock suction/irrigators separately and only open one up if it is needed during each surgery. In this case, they can open one up and quickly attach it to the tube set during a surgery if it is needed.

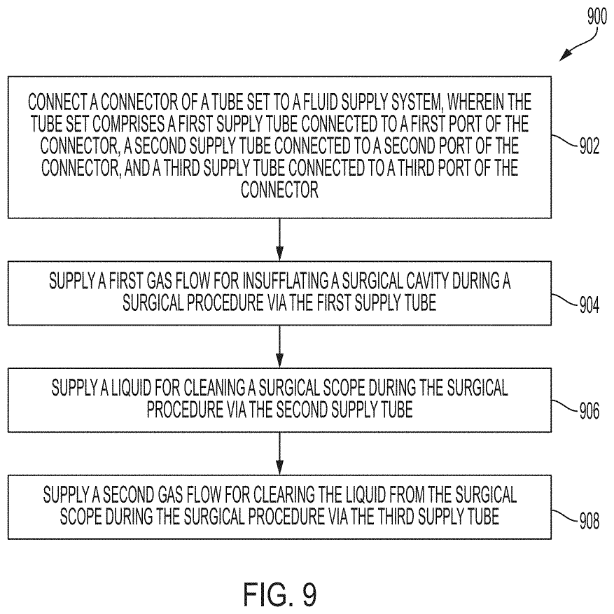

FIG. 9 illustrates a method 900 for supplying fluid to a surgical field, According to various aspects. At step 902, a connector of a tube set is connected to a fluid supply system. The connector can be, for example, any of connectors 410, 510, or 610 and the fluid supply system can be any of apparatus 400, 500, or 600. The tube set can be, for example, tube set 700 of FIG. 7. The tube set includes a first supply tube connected to a first port of the connector, a second supply tube connected to a second port of the connector, and a third supply tube connected to a third port of the connector. For example, with reference to FIG. 4, the tube set can include insufflating gas line 428 connected to the insufflating gas supply port 426, liquid supply line 418 connected to the liquid supply port 416, and the gas supply line 414 connected to the gas supply port 412.

At step 904, a first gas flow for insufflating a surgical cavity is supplied during a surgical procedure via the first supply tube. For example, carbon dioxide can be supplied via the insufflating gas line 428 of FIG. 4 for insufflating the surgical cavity. At step 906, a liquid for cleaning a surgical scope is supplied during the surgical procedure via the second supply tube. For example, saline or a saline solution can be supplied via the liquid supply line 418 of FIG. 4 to a surgical scope cleaner, such as scope cleaner 100. At step 908, a second gas flow for clearing the liquid from the surgical scope is supplied during the surgical procedure via the third supply tube. For example, carbon dioxide can be supplied via the gas supply line 414 to the surgical scope cleaner.

Optionally, the method 900 further includes, prior to connecting the connector to the fluid supply system, unpackaging the tube set, which has been pre-sterilized and packaged. Optionally, the packaged tube set includes the scope cleaner. Alternatively, the method 900 further includes attaching the second and third supply tubes that are connected to a surgical scope before or after the tube set connector is connected to the fluid supply system. Optionally, the method 900 includes discarding the tube set after use for a single surgical procedure. Alternatively, the method 900 includes re-sterilizing the tube set after use.

Optionally, the method 900 further includes evacuating the surgical cavity via an evacuation tube connected to a fourth port of the connector. For example, smoke from the surgical cavity can be evacuated via a smoke evacuation line 432 connected to a smoke evacuation port 430 of connector 410 that is connected to apparatus 400.

Optionally, gas flow via the surgical scope cleaner can be used to supply a second insufflation gas supply means. Occasionally, during a surgical procedure, a leak will occur that will not allow the fluid supply management apparatus to push enough gas into the surgical cavity to maintain the desired pneumoperitoneum pressure. The scope cleaning sheath can be utilized as a second source of gas from the fluid management apparatus to provide enough flow rate to overcome the leak and keep the surgical cavity pressurized in these extreme leak scenarios.

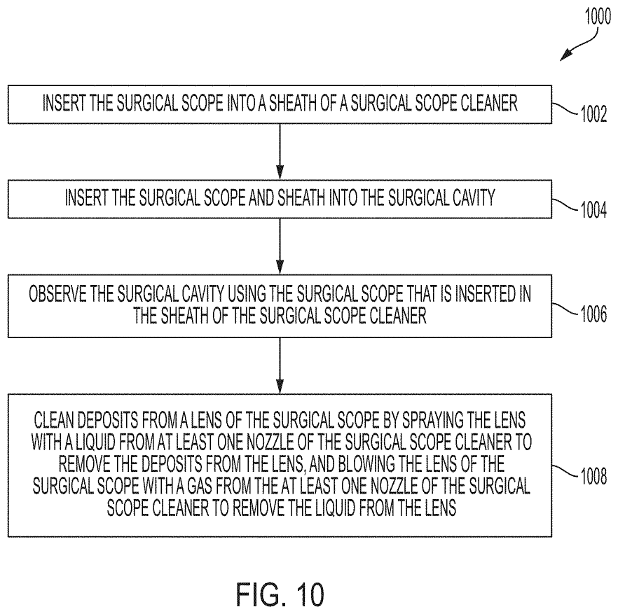

FIG. 10 illustrates a method 1000 for cleaning a surgical scope, optionally while the surgical scope is inserted, or pre-inserted, in a, e.g. surgical, cavity, According to various aspects. At step 1002 the surgical scope, such as scope 150 of FIG. 1A is inserted into a sheath of a surgical scope cleaner, such as sheath 102 of scope cleaner 100. At optional step 1004, the surgical scope and sheath are inserted, or pre-inserted, into the, e.g. surgical cavity. For example, with reference to FIG. 1A and FIG. 7, the tube 152 of the surgical scope 150 with the mounted sheath 102 of the cleaner 100 are inserted, or pre-inserted, through the lumen of a trocar 730 into the, e.g. surgical, cavity 750. Alternatively, the surgical scope is not inserted or pre-inserted into the, e.g. surgical, cavity. The method may for example exclude physical intervention on the human or animal body. At optional step 1006, the, e.g. surgical cavity is observed using the surgical scope that is inserted, or pre-inserted, in the sheath of the surgical scope cleaner. For example, images generated by endoscopic camera 746 of FIG. 7 can be displayed via a display in the operating room and observed by the user, e.g. the surgeon.

At step 1008, deposits from a lens of the surgical scope may be cleaned by spraying the lens with a liquid from at least one nozzle of the surgical scope cleaner to remove the deposits from the lens, and blowing the lens of the surgical scope with a gas from the at least one nozzle of the surgical scope cleaner to remove the liquid from the lens. For example, with reference to FIG. 1C, deposits from lens 156 of the surgical scope 150 may be cleaned by spraying the lens 156 with saline from nozzle 112 of the surgical scope cleaner 100 to remove the deposits from the lens 156 and then blowing the lens 156 of the surgical scope 150 with a burst of carbon dioxide from nozzle 114 of the surgical scope cleaner 100 to remove the saline from the lens 156. Each of the liquid spray and the burst of gas can be provided for pre-determined periods of time that may be the same length or different lengths. The respective periods of liquid spray and burst of gas can overlap such that liquid and gas is provided simultaneously for at least a portion of the time.

Optionally, the cleaning sequence described above can be performed in response to a user command. For example, a user may see blurring on one or more endoscopic images or video displayed on the display in the operating room indicating smudging and/or fogging of the lens of the scope and may issue a command to commence the scope cleaning sequence. The command may be provided, for example, via a button press on the endoscopic camera, such as endoscopic camera 746 of FIG. 7. The button press can be communicated via communication line 744 to a camera controller, such as camera controller 810 of FIG. 8B. The camera controller can be communicatively connected to a fluid management apparatus. For example, with reference to FIG. 3, the camera controller can be a component of external system 334 or communicatively connected to external system 334, which is communicatively connected to fluid management apparatus 300. Based on the user's command, the external system 334 can send a command to the apparatus 300 to perform the cleaning sequence. The user could also push a button he/she temporarily attaches to the scope, the camera head, or that comes integrated into the proximal end of the scope cleaning sheath with an electrical wire running to the insufflator via the connector and said button/switch would be integrated into the tube set.

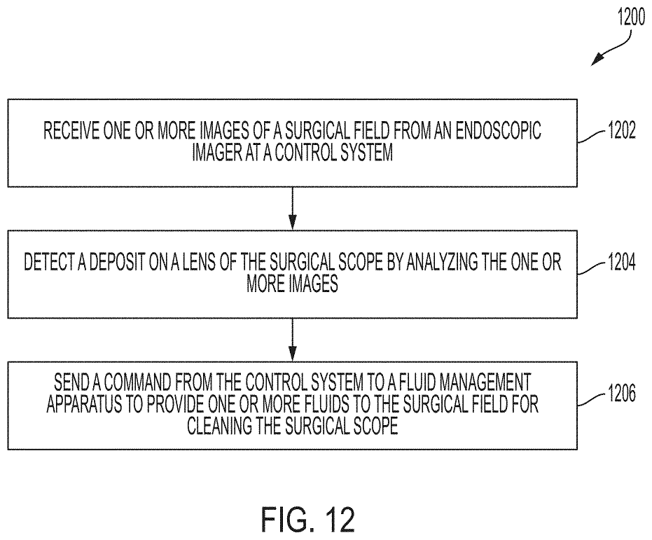

Optionally, an image analysis and control system, such as external system 334, includes image processing that analyzes one or more images or one or more video frames generated by the endoscopic imager to detect scope smudging and/or fogging. Once the scope smudging and/or fogging has been detected, the control system may send a control command to the fluid management apparatus 300 to perform a cleaning sequence. Accordingly, Optionally, the control system includes one or more processors and memory storing one or more programs for performing a method to automatically detect deposits on a lens of a surgical scope and send a command to a connected apparatus to initiate a cleaning sequence for the surgical scope. An exemplary method performed by a control system, according to various aspects, is method 1200 of FIG. 12. At step 1202 of method 1200, the control system receives one or more images of a, e.g. surgical, field from an endoscopic imager that is communicatively connected to the control system. Optionally, at step 1202 of method 1200, the control system receives one or more images of a, e.g. surgical, field from a pre-inserted endoscopic imager that is communicatively connected to the control system. The endoscopic imager includes an endoscopic camera connected to a scope, such as surgical scope 150, that is optionally inserted or pre-inserted in the surgical cavity. The scope may be received in a scope cleaner, such as surgical scope cleaner 100, or may have integrated cleaning functionality, such as scope 1100 of FIGS. 11A-E. At step 1204, the control system automatically detects a deposit on a lens of the surgical scope by analyzing the one or more images. Any suitable image processing algorithm or combination of algorithms may be used to detect deposits. At step 1206, the control system sends a command to a communicatively connected fluid management apparatus to provide one or more fluids, e.g. to the surgical field, for cleaning the surgical scope. Optionally, the command may be sent automatically in response to detecting deposits. According to various aspects, in response in response to receiving the command from the control system, the fluid management apparatus supplies a liquid flow and/or a gas flow from the apparatus to the surgical scope cleaner or surgical scope with integrated cleaning for cleaning a lens of the surgical scope, in particular during a surgical procedure. Optionally, the liquid flow is supplied first for a first period and the gas flow is supplied for a second period that is at least partially subsequent to the first period.

Optionally, the image analysis and control system may provide a notification to the user, such as on a display in the operating room, that smudging and/or fogging has been detected. The image analysis and control system may wait for a confirmation from the user to initiate the cleaning sequence. The user may confirm that the cleaning sequence may be performed via any suitable user input, such as a voice command, a button press on the camera head, or a button press on a user interface of the image analysis and control system. In response to receiving the user command, the image analysis and control system may send an initiate cleaning sequence command to the fluid management apparatus, which may respond by controlling the cleaning sequence. Optionally, the control system may wait for a predetermined period of time (which can be configurable) before initiating scope cleaning.

The fluid cleaning apparatus may control a cleaning sequence by actuating the actuator to provide flow of the liquid to the scope cleaner. For example, the controller 328 of apparatus 300 may send a command to actuator 316 to cause the flow device 318 to permit the flow of liquid received from the reservoir 318. With reference to FIGS. 4C and 4D, this may include controlling the solenoid 448 so that the plunger 446 moves the lever 442 to cause the valve 438 to move to the open position. Optionally, the cleaning sequence includes spraying the lens with liquid for a first predetermined period. Once this period has elapsed, the actuator may be controlled to stop the flow of liquid. For example, the plunger 446 may be retracted and the valve 438 may return to a closed position due to the force of the spring 444.

The fluid cleaning apparatus may continue the cleaning sequence by opening a valve for pressurized gas to flow to the surgical scope cleaner. For example, with reference to FIG. 3, the controller 328 may control the valve 314 to open, allowing pressurized gas from gas supply inlet 306 to flow (as regulated by, for example, regulator 312 or regulator 315) to the scope cleaner. The gas may be provided for a second predefined period of time. The burst of gas may be provided while the liquid is being provided or may be provided entirely after the liquid is provided.

Various scope cleaning sequence parameters may be configurable and adjustable by the user, such as before and/or during and/or after a surgical procedure. For example, one or more of the following parameters could be configurable by the user (and could include default settings): length of time the washing fluid is sprayed onto the lens, length of time the gas is directed across the lens after washing to clean the lens, pressure (and in turn the flow rate) of the washing fluid, pressure (and in turn the flow rate) of the gas directed across the lens after washing, and whether or not to include a fluid washing step in the cleaning sequence (some might prefer to just have the gas blow the debris off the lens, even if it isn't perfectly clean, because this option does not ever cause a disruption of the surgical image).

According to various aspects, surgical scope cleaning is built into the surgical scope itself by building at least one fluid channel and at least one fluid outlet into the scope shaft. This can be particularly advantageous for small surgical scopes, such as sinuscopes, for which a separate cleaning sheath may be prohibitively large for inserting into narrow passageways, such as in the sinuses. Thus, according to various aspects, the scope and cleaning sheath functions are combined into a single solution—a scope having integrated cleaning capability. By combining these two conventionally separate functions, the overall size of the scope with cleaning capability can be minimized and a much smaller cross-sectional area can be achieved than a separate scope and sheath solution. According to various aspects, integration of the cleaning solution into the scope has other advantages, including reducing the amount of reflections and other visual impairments (obstruction of view, etc.) that are introduced by a separate sheath and maintaining the working length of the scope, which would otherwise be shortened by a sheath.

A scope with integrated cleaning, according to various aspects, can be particularly suitable for functional endoscopic sinus surgery (FESS) and Transnasal Skull Base surgeries for which the cross-sectional size of the inserted device is a major design limiter due to the limited size of the operating space. According to various aspects, by integrating the cleaning channel into the scope, the size of the combined solution can be as small as an elliptical cross section with a height of 4.6 mm and width of 4.0 mm (which is the size of a conventional sinuscope) while maintaining a cleaning channel cross-section that is sufficiently large for use with pumps and tubing that are conventionally used in the operating room.

FIGS. 11A-E illustrate a scope having integrated cleaning functionality, According to various aspects. Looking first at FIG. 11A, scope 1100 includes a shaft 1102 that extends distally from a main body and is configured for insertion into a surgical cavity during use. The shaft 1102 includes at least one fluid channel (described further below) that directs fluid to at least one fluid outlet 1114 at the distal end 1116 of the shaft 1102. The at least one fluid outlet 1114 is configured to direct fluid onto an optical component 1112 (such as a window or lens) located at the distal end 1116 of the shaft.

The main body 1104 includes an eyepiece 1106 located at a proximal end 1118 of the scope 1100. The eyepiece 1106 can be configured for connecting the scope 1100 to an imager. The main body includes a light port 1108, which can be configured as a light cable connector for connecting to a light cable that provides illumination to the scope 1100. The main body 1104 also includes at least one fluid port 1110 for connecting to at least one fluid supply and/or exhaust supply system for supplying fluid and/or exhaust to the scope 1100. The at least one fluid port 1110 can be configured for a liquid, such as saline, or for a gas, such as carbon dioxide (as used herein, the term “fluid” encompasses liquids and gases). Optionally, the main body 1104 includes a single fluid port 1110. Optionally, the main body 1104 includes multiple fluid ports 1110, such as a liquid port and a gas port. Optionally, a fluid port 1110 can be used to supply both a liquid and a gas, either sequentially (such as via upstream valving) or simultaneously (such as to increase the pressure of supplied liquid.) Optionally, fluid flows both into and out of the port 1110, such as due to a peristaltic operation of a fluid supply system. The at least one fluid port 1110 can be configured for connecting to conventional tubing used for supplying fluids to the surgical field.

FIG. 11B illustrates a perspective view of a cross-section (taken on line A-A of FIG. 11A) of a portion of the shaft 1102 of scope 1100. The shaft 1102 includes at least one fluid channel 1120 for fluid to flow between the at least one fluid port 1110 in the main body 1104 and the at least one fluid outlet 1114 at the distal end 1116 of the shaft 1102. The at least one fluid channel 1120 is located between a first wall portion 1122 that corresponds to the outer tubular wall of a conventional scope shaft (see tube 152 of scope 150 of FIGS. 1A-1B for an example of an example of an outer tubular wall of a conventional scope configuration) and a second wall portion 1130 that extends at least partially around the first wall portion 1122.

According to various aspects, the second wall portion 1130 extends only partially around the first wall portion 1122 such that the external surface of the shaft 1102 is formed by the second wall portion 1130 and the portion of the first wall portion 1122 that is not surrounded by the second wall portion 1130. With the second wall portion 1130 extending only partially around the first wall portion 122, the outer surface of the shaft 1102 is non-cylindrical. The increase in size needed to accommodate the at least one fluid channel 1120 is concentrated in a width 1134 of the shaft 1102 in the direction of the major axis, with the increase in width 1132 in the direction of the minor axis being less or none at all relative to the shaft of a conventional endoscope of the same size. Optionally, the first wall portion 1122 is cylindrical and the width 1132 of the shaft 1102 in the direction of the minor axis is equal to the diameter of the first wall portion 1122, such that there is no increase in width of the shaft 1102 along the minor axis relative to a conventional scope shaft of the same size. For example, the width 1132 along the minor axis for an endoscope 1100 sized to correspond to a conventional 4 mm scope may be 4 mm.

According to various aspects, the first wall portion 1122 and second wall portion 1130 are integrated into a unitary piece, which can be formed in any suitable fashion, such as via welding the second wall portion 1130 to the first wall portion 1122, extrusion, and/or machining. Optionally, multiple fluid channels are provided between the first wall portion 1122 and the second wall portion 1130, such as configured like the two conduits 118 and 120 shown in FIG. 1D.

The shaft 1102 includes an inner tube 1124 that is located radially inwardly of the first wall portion 1122 and defines with the first wall portion 1122 a channel 1126 for locating fiber optics that carry light from a light cable connected to the light port 1108. The inner tube 1124 defines an optical channel 1128 for directing light from a scene and can house one or more optical components (not shown).

FIGS. 11C and 11D are perspective and cross-sectional views, respectively, of a distal portion of the shaft 1102, according to various aspects. At least one fluid outlet 1114 is provided at the distal end 1116 of the shaft 1102 and is configured for directing fluid from the at least one fluid channel 1120 onto an optical component 1112 located at the distal end of the 1116 of the shaft 1102. The at least one fluid outlet 1114 is configured to turn the fluid flow so that it impinges on the optical component 1112 to wash and/or blow deposits from the optical component 1112. Optionally, the at least one fluid outlet 1114 is formed by rolling a distal end of the second wall portion 1130 inwardly. Optionally, a separate fluid outlet 1114 is joined to the distal end of the second wall portion 1130. Optionally, the at least one fluid outlet is rigidly disposed on the shaft 1102 to ensure that the at least one fluid outlet does not obscure the field of view of the endoscope 1100. The at least one fluid outlet can be configured to minimize reflections, such as by being provided with a non-reflective coating or formed of a non-reflective material.

Optionally, a fluid outlet can be provided for each of multiple fluid conduits. For example, multiple fluid outlets could be configured as in nozzle head 110 of FIG. 1C, which includes two nozzles 112, 114. Optionally, a single fluid outlet can be provided for multiple fluid conduits. The single fluid outlet can be configured for providing fluids from the multiple conduits sequentially— such as a liquid spray followed by a gas blow—and/or for providing a mixture of the fluids from the multiple conduits.

FIG. 11E is a cross section of a proximal portion of the shaft 1102 and a portion of the main body 1104, According to various aspects. The at least one fluid port 1110 communicates with the at least one fluid channel 1120 of the shaft 1102 via a fluid passageway 1136 in the main body 1104. Optionally, the fluid passageway 1136 is defined by a gap between the first wall portion 1122 and an opening in the main body 1104 formed for receiving the first wall portion 112.

The second wall portion 1130 can be sealed to the main body 1104 to prevent fluid leakage, such as by welding the second wall portion 1130 to the main body 1104. Optionally, the second wall portion 1130 terminates at the main body 1104.

According to various aspects, the first wall portion 1122 extends into the main body 1104 and terminates within the main body 1104. Fiber optics 1138 from the light port 1108 extend into the channel 1126. The inner tube 1124 may extend toward the proximal end of the main body 1104, terminating at the eyepiece 1106.

According to various aspects, to prevent fluid leakage into the optical portion of the main body 1104, a seal 1140 is positioned in the main body 1104 for sealing between the main body 1104 and the outer surface of the first wall portion 1122 at a location that is between the fluid port 1110 and the light port 1108. The seal 1140 can prevent fluid from flowing proximally into the light port portion of the main body 1104.

Endoscope 1100 can be used according to any of the methods described above, including method 900 of FIG. 9 and method 1000 of FIG. 10, except that the fluid flow and nozzles are provided directly on the endoscope 1100 rather than as part of a sheath. For example, according to various aspects, one or more tubes of one or more fluid supply systems, such as apparatus 300 for managing fluid flow into and out of a surgical field of FIG. 3, fluid supply apparatus 400 of FIGS. 4A and 4B, fluid supply apparatus 500 of FIGS. 5A-5B, or fluid supply apparatus 600 of FIG. 6, are connected to the one or more fluid ports 1110 of the endoscope 1100. This may be done, for example, using tube set 700 of FIG. 7. The endoscope 1100 is then optionally inserted or pre-inserted into the patient's body. Due to the smaller size of the shaft 1102 of the endoscope 1100 relative to a cleaning sheath for the same relative size scope, smaller spaces and/or a smaller incision (for a smaller trocar) can be achieved. The endoscope 1100 can be used in a conventional manner and when deposits form on the optical component 1112, one or more fluids can be directed to the optical component 1112 to remove the deposits. The triggering of the fluid flow can be achieved in any suitable manner, including according to any of the methods described herein for sheath-based cleaning.

According to various aspects, the endoscope 1100 is configured as a sinuscope and has a single fluid port 1110, single fluid channel 1120, and single fluid outlet 1114. The endoscope 1100 is attached to commercially available sinuscope cleaning pump(s) via tubing connected to the fluid port 1110. When the pump is activated, fluid (such as saline) flows into the fluid port 1110, flows through the fluid channel 1120, and flows onto the optical component 1112 of the endoscope 1100 to wash deposits (such as smudging or fogging) from the optical component 1112. During a reverse cycle of the pump(s), fluid can be drawn back into the fluid channel 1120 via the fluid outlet 1114 (which then functions as a fluid inlet) to remove fluid from the optical component 1112 of the endoscope 1100. According to various aspects, the cross-sectional area of the fluid channel 1120 is optimized to prevent the development of back-pressure in the tubing while also allowing for appropriate velocity and direction of the fluid at the distal end 1116 of the endoscope 1100.

As described above with respect to the surgical scope cleaner 100 of FIG. 1A, the liquid and gas flow paths merge at or near the liquid and gas ports 106, 108 such that a single conduit extends substantially the entire longitudinal extent of the sheath 102. An example of this arrangement is illustrated in FIGS. 13A and 13B. Surgical scope cleaner 1300 includes a sheath 1302 that has a single conduit 1304 through which both liquid and gas can flow to a single nozzle located at the distal end of the cleaner 1300. This configuration can be advantageous over a two-conduit arrangement such as that of sheath 102 of FIG. 1A by allowing for the sheath to be smaller in diameter (e.g., for the same flow rate). As shown in the cross section of the sheath 1302 in FIG. 13A, the single conduit 1304 extends longitudinally through the sheath 1302 and is formed as a longitudinally extending cavity within wall 1306. The conduit 1304 can be non-circular in cross-section and can curve about the longitudinal axis 1308 of the sheath 1302. The curved cross-sectional shape of the conduit 1304 enables the outer diameter of the wall 1306 to be minimized while still providing a sufficient flow rate through the conduit 1304. Alternatively, the conduit may be circular or any other suitable shape depending on the wall thickness and the desired flow rate and pressure drop through the conduit.

The scope cleaner 1300 includes a liquid port 1310 and a gas port 1312. The liquid and gas pathways from the ports 1310, 1312 merge downstream of the liquid and gas ports 1310, 1312 at a merge site 1314 into a single flow path 1316 that leads to the single conduit 1304 in the sheath 1302. Optionally, a valve mechanism 1318 may be included to prevent liquid from being pulled into the gas flow and to prevent gas from being pulled into the liquid flow. The valve mechanism 1318 can be located at the merge site 1314 to shut off the supply of one while the other one is flowing. This arrangement can help eliminate the Venturi effect, which undesirably pulls some liquid into the conduit of the sheath while the gas is flowing and can make it difficult to fully dry the lens of the scope. The illustrated valve mechanism 1318 is a small shuttle valve that includes a movable component 1320 that shuttles back and forth between the gas and liquid pathways based on whichever pathway has the highest pressure at the time. So, for example, when pressurized saline is provided to the liquid port 1310 while the gas supply to the gas port 1312 is turned off, the movable component 1320 is pushed over to close off the gas line based on the pressure differential and is pushed against a sealing member 1322. In the illustrated example, the moveable component 1320 is a spherical ball and the sealing member 1322 is an O-ring. However, other sealing member shapes and types can be used, including shuttle valve 238 of FIG. 2. The valve mechanism 1318 allows the liquid to flow through the sheath without any gas mixture. When the liquid supply is stopped and the gas supply is provided, the moveable component 1320 will be pushed back onto a sealing member 1324, which will allow gas to flow through the sheath without any liquid getting mixed with it.

As described above, a fluid management system such as apparatus 300 of FIG. 3 can provide gas to a surgical site, such as for insufflation. This gas can either be supplied through a system of pipes in the hospital or surgery center, or it can come from one or more high-pressure tanks (also referred to as bottles) of gas located in the operating room. When a high-pressure tank is used in the operating room, it is problematic when the gas supply runs out during a surgical procedure, especially if it causes a reduction or a loss of pressure inside the surgical cavity, which can cause unsafe conditions for the patient if the pressure drops low enough such that the surgeon suddenly loses the ability to see what he/she is doing or does not have enough workspace to perform a critical action, such as suturing up a bleeding vessel, etc. This problem can be exacerbated by the advent of better smoke evacuation during these procedures to remove and filter out the smoke that is created by a surgeon's frequent use of electrosurgical tools and instruments for cutting, cauterizing, and sealing. In order to evacuate the smoke, a supply of gas is pumped into the surgical cavity to replace the displaced or evacuated smoke-filled gas. Oftentimes, the surgeon will use smoke evacuation at a rate of 9 to 12 liters per minute during the surgery, and sometimes for several hours. At 12 liters per minute, a two-hour surgery could use about 1,440 liters of gas. A typical size used for bottled carbon dioxide in operating rooms is the E size cylinder (being filled with up to 1,600 liters), meaning that a surgeon might go through a bottle in just one surgery, depending on the length of the surgery and whether or not the smoke evacuation was used during the entire surgery.

In some surgery centers, a pressure gauge is used to monitor the pressure of the gas inside each tank, however this is normally not the case. Normally, the surgical staff waits for a notification from the insufflator telling them that the tank currently being used is running low on pressure. There is normally inferior detection of the gas pressure becoming low until shortly before it actually becomes empty, because the surgical staff is occupied with many things during the surgery and is not normally looking at the pressure gauge or the gas pressure indicator on the insufflator.

Described in detail below is an automatic gas supply switching system that monitors the availability of gas in gas tanks in the operating room and automatically switches away from an empty tank to a full (or fuller) tank to ensure the continuous supply of gas during surgery and to free the surgical staff from worrying about the pressure in the gas tanks during surgery, enabling them to remain focused on the surgery and the patient. Additionally, the system can be configured to alert support staff within the surgery center that an empty tank needs to be replaced, such as after the surgical procedure is completed and the room is being cleaned and prepared for the next surgical procedure.

The automatic gas supply tank switching system can include a valve assembly connected to the insufflation gas flow circuit on the outflow side and to at least two separate tanks of gas (e.g., in parallel) on the inflow side. The system can monitor one or more properties of the gas supply from the tanks of gas during the course of the surgical procedure. The valve assembly can allow flow of gas from one tank or set of tanks, while maintaining the other tank(s) of gas closed. When the flow of gas becomes insufficient (e.g., based on a measured pressure and/or flow rate), the system can automatically shut off the flow path from the empty tank(s) and open the flow path from a fuller tank or set of tanks. The valve assembly could be, for example, a two-way valve, such as a ball valve, that switches back and forth between two supply lines, or an alternative approach includes having two or more separate valves (one for each tank of gas or each set of gas tanks). The system could alert staff about the empty tank and indicate which of the tanks requires replacement.

Optionally, the system monitors pressure of the supply tanks, such as via a pressure transducer that supplies an output voltage level, which can be converted by a controller to a pressure based on the relationship between voltage and pressure from the calibration curve of the transducer. The controller can then send an electrical signal to either a solenoid or a motor that is used to operate the valve(s). In the case of one two-way valve switching between the two gas tanks, the solenoid or the motor is used to provide either a rotational load to turn a ball valve or a translational load to push a piston from one side to the other. Since the gas pressure in the tanks can be very high (for example, as much as 80 bars or 1,160 psi), the solenoid or the motor can be coupled with a means of obtaining a mechanical advantage, such as a gear train or a lever to overcome the load caused by the high pressure differential to hold a full tank of gas closed or sealed. Optionally, the system could include a sensor or sensors to make sure that the valve has properly closed off the empty tank and opened the full tank. The sensor(s) could include one or more positional sensors to detect the position of a valve, and/or one or more pressure and/or flow sensors to detect whether the valve is opened or closed based on properties of the fluid flow. If the system determines that the empty tank is not completely sealed off or that the full tank is not fully opened, then the system can continue to drive the solenoid or the motor until proper valve positioning is determined.

In some variations, pressure measurement is purely mechanical in nature, taking advantage of the pressure differential between the gas lines to create a force large enough to drive the valve mechanism, which opens one line and closes the other. This can be practical since the actual pressure reading of each tank of gas may not be important to the surgical staff. In this variation, the valve mechanism could be configured to actuate when a certain pressure differential is present, such as to avoid closing off a tank that is not empty. For example, a spring-loaded sliding or rotating mechanism can be used to operate the valve between the two tanks of gas that requires a pressure differential of, say, 55 bars (800 psi) before enough force can be generated to actuate the mechanism. The valve mechanism could be configured to indicate which tank is empty by, for example, uncovering a placard, label, or tag on one side of the valve and covering up an opposing placard, label, or tag on the other side of the valve, so that quickly glancing at the valve assembly makes it easy to see which tank is full and which is empty.

The valve assembly can be located near the tanks and connected to them through high-pressure hoses or can be mounted to, housed within, and/or integral to the fluid delivery apparatus (e.g., apparatus 300 of FIG. 3). High-pressure hoses could extend from the fluid delivery apparatus to the tanks of gas. Each of the two high-pressure hoses could have an identifying means about them, such as being two different colors, or having placards or labels or tags on them so that tank 1 and tank 2 (or tank A and tank B) can be identified by just a quick glance. The system could provide a visual indication that shows the status of each of the two tanks of gas and could provide an alert (visual and/or audible) that warns the staff about the empty tank.

In some variations, the system can communicate with an external system, such as a medical room controller, and provide the external system with an empty tank status so that the surgical staff can be alerted, such as through a message appearing on the surgeon's display (the monitor that the surgeon and the surgical staff are looking at during surgery). The external system could be connected to or include the network of the hospital or surgery center, and an alert can also be sent to others outside of the operating room so that the surgical staff inside the operating room does not have to become distracted with the alert of an empty tank. Someone else in the hospital or surgery center can come and change the empty tank at their convenience. Notification to others outside of the operating room can be through a text message, an email, a beeper, or an audible or visual alarm or indicator, or any other suitable means.

Optionally, the system can detect whether or not gas is leaking from a gas tank due to a faulty connection (for example, a hose not tightened well enough or a leak in a high-pressure connection within the fluid delivery apparatus) and can automatically switch over to another tank (and then alert the surgical staff and/or to others outside of the operating room to this problem). For example, the system may track how much gas is being consumed, compare this to the rate at which the pressure in the tank should be dropping over time, and detect leaking by determining that the pressure is dropping at a faster rate than it should be.

In variations in which the automatic gas supply tank switching system is not part of the fluid delivery apparatus, the switching system could be a stand-alone device that works autonomously or can be communicatively connected to the fluid delivery apparatus either wirelessly (e.g., Bluetooth, WiFi) or through a connecting cable. A connecting cable could be used to also supply power to the switching system.

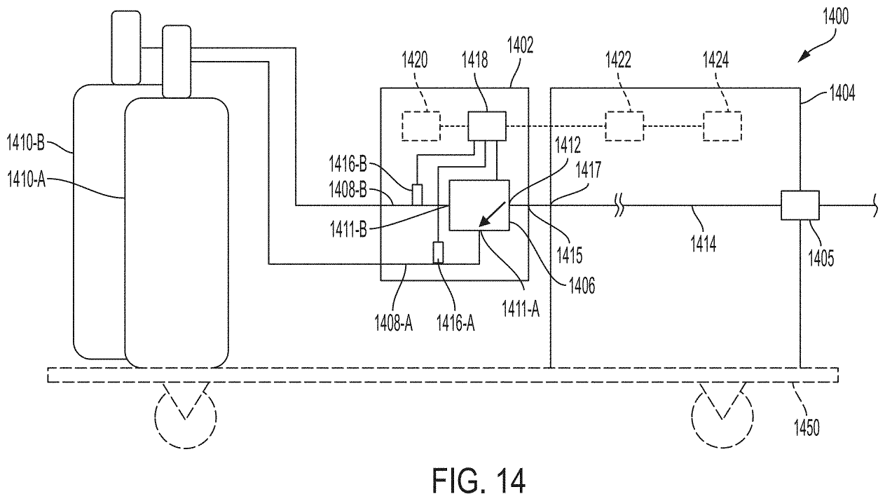

FIG. 14 is a block diagram of a fluid delivery system 1400 that includes an automatic gas supply tank switching system 1402 for automatically switching gas supply sources to ensure a continuous supply of gas to a gas supply apparatus 1404, which provides gas to a surgical field via a gas outlet 1405. The automatic gas supply tank switching system 1402 can be a standalone device that is separate from the fluid delivery apparatus 1404 (for example, located at the tanks, located on the same cart 1450 as the apparatus 1404, or mounted to the fluid delivery apparatus 1404) or can be integrated into the fluid delivery apparatus 1404.

The switching system 1402 includes a valve assembly 1406 that can switch between gas supply via a first supply line 1408-A and gas supply via a second supply line 1408-B. The valve assembly 1406 can include a single two-way valve, such as a shuttle valve or ball valve, that is in fluid communication with both supply lines, or can include a plurality of one-way valves, such as one for each supply line. In some variations, the valve assembly 1406 can switch between more than two supply lines. The gas supply lines 1408-A and 1408-B can be fluidly connected to at least two gas supply tanks 1410-A and 1410-B that are located in the same operating room (e.g., on the floor adjacent to the gas supply apparatus 1404 or on the same cart 1450). The gas supply lines 1408-A and 1408-B can be or include hoses connected at one end to a manual valve of a respective gas tank and at the other end to a respective inlet 1411-A and 1411-B of the gas supply tank switching system 1402 or valve assembly 1406. In some variations, each supply line is connected to multiple gas supply tanks, such as via multiple hoses connected at a respective manifold. The valve assembly 1406 includes an outlet 1412 fluidly connected to the gas flow circuit 1414 of the gas supply apparatus 1404 either directly (e.g., when the switching system 1402 is integrated into the fluid supply apparatus 1404) or via an outlet 1415 of the switching system 1402 fluidly connected to an inlet 1417 of the fluid supply apparatus 1404.

In some variations, one or more sensors 1416-A and 1416-B sense one or more aspects of the gas supply via the gas supply lines 1408-A and 1408-B. The sensors 1416-A and 1416-B can include, for example, pressure sensors and/or flow rate sensors. A controller 1418 can receive signals from the sensors 1416-A and 1416-B and can determine based on the signals that the valve assembly 1406 should switch to a different supply line. For example, a controller 1418 can determine that the pressure in supply line 1408-A as sensed by pressure sensor 1416-A is below a predetermined threshold and can control valve assembly 1406 to switch from permitting flow from supply line 1408-A to permitting flow from supply line 1408-B (and blocking flow from supply line 1408-A). Flow rate could also be used to trigger actuation of the valve assembly 1406, such as when the flow rate drops below a predetermined threshold. Flow rate could also or alternatively be used to determine that the valve assembly 1406 has properly shut off flow via one supply line and/or has properly opened flow via the other supply line. For example, the controller 1418 could command actuation of a valve until the flow associated with the valve reduced below a predetermined threshold (for closing) or until flow rises above a predetermined threshold (for opening).

In variations in which the valve assembly 1406 is a single valve, the controller 1418 may send a signal to the single valve to actuate the single valve, which through its actuation closes off a flow path for one supply line and opens a flow path for the other supply line. In variations in which multiple valves are used, the controller 1418 could command a first valve associated with the empty supply tank to close and command a second valve associated with the full supply tank to open.

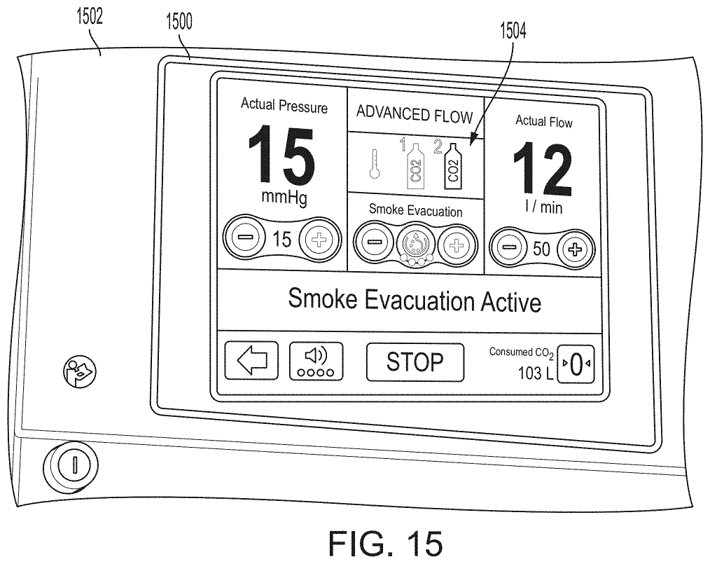

The controller 1418 could be a controller for the switching system 1402, such as in variations in which the switching system 1402 is not integrated into the fluid supply apparatus 1404, or could be a controller of the fluid supply apparatus 1404. The controller 1418 can be connected to a display 1420, such as one or more LEDs or a display screen, for providing an indication related to the status of the supply tanks 1410-A, 1410-B. The display 1420 could be a display for the switching system 1402, or the controller 1418 of the switching system 1402 could communicate with a controller 1422 of the fluid supply apparatus 1404, which could provide a notification related to tank status on a display 1424 of the apparatus and/or could provide a notification to an external system, such as a medical room control or monitoring system. FIG. 15 illustrates an exemplary display 1500 of a fluid supply apparatus 1502 that displays visual indicators 1504 of the statuses of two gas supply tanks.

Optionally, the valve assembly 1406 switches between supply lines based on a pressure differential defined by a mechanical configuration of the valve assembly 1406. For example, the valve assembly 1406 may include a spring-loaded piston that actuates to switch between supply lines when a pressure differential across the piston reaches a predetermined threshold.

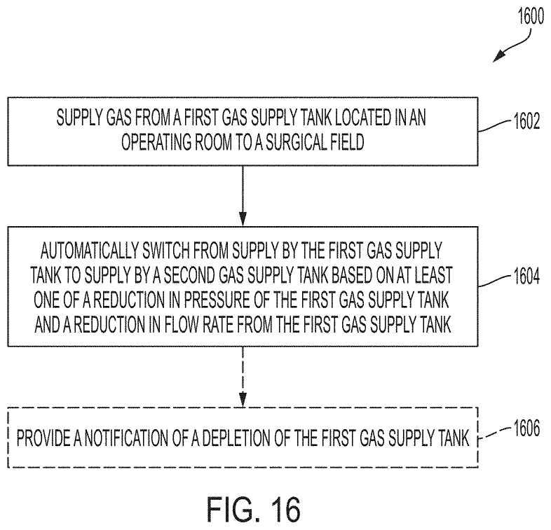

FIG. 16 illustrates a method 1600 for supplying gas, such as insufflation gas, for example for a surgical procedure. Method 1600 may exclude physical intervention on the human or animal body. Method 1600 can be performed by, for example, system 1400 of FIG. 14. At step 1602, gas is supplied from a first gas supply tank located in an operating room to a field, particularly a surgical field. At step 1604, supply of gas is switched from supply by the first gas supply tank to supply by a second gas supply tank based on at least one of a reduction in pressure of the first insufflation gas supply tank and a reduction in flow rate from the first insufflation gas supply tank. For example, a pressure of the first gas supply tank can be monitored and a valve can be automatically actuated to switch the supply in response to determining that a pressure of the first gas supply tank is below a predetermined threshold. Pressure and/or flow sensors may be used to monitor the availability of gas supply of the respective tanks and a controller can switch to a different supply tank based on analysis of the measurements from the pressure and/or flow sensors. Alternatively, supply could be automatically switched by a valve that automatically actuates based on a pressure differential.

Method 1600 can include optional step 1606 in which a notification of a depletion of the first gas supply tank is provided. Optionally, the notification indicates which of the at least two gas supply tanks is depleted.

Method 1600 can also include monitoring an amount of gas provided to the, e.g. surgical, field and detecting a gas supply leak by comparing the amount of gas provided to the, e.g. surgical, field to a drop in pressure of at least one of the first and second gas supply tanks. For example, a drop in pressure that appears to be greater than would be expected for the given gas supply to the surgical field can be used to determine that there is a leak in the flow path(s). A notification could be provided to a user (in the operating room or elsewhere) that the system should be checked for leaks.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A system for supplying insufflation gas for a surgical procedure comprising:
first and second insufflation gas inlets for receiving insufflation gas from at least two insufflation gas supply tanks located in an operating room;
an insufflation gas outlet for providing a flow of insufflation gas supplied via the first and second insufflation gas inlets; and
a valve system configured to automatically switch from insufflation gas supply via the first insufflation gas inlet to insufflation gas supply via the second insufflation gas inlet to maintain insufflation gas flow at the insufflation gas outlet.

2. The system of claim 1, further comprising a sensor system for detecting at least one pressure, at least one flow rate, or at least one of both pressure and flow rate that is associated with the at least two insufflation gas supply tanks, wherein the valve system comprises at least one valve and a control system configured to control the at least one valve to switch from insufflation gas supply via the first insufflation gas inlet to insufflation gas supply via the second insufflation gas inlet.

3. The system of claim 1, wherein the valve system comprises a two-way valve in fluid communication with the first and second insufflation gas inlets.

4. The system of claim 1, wherein the valve system comprises at least two one-way valves.

5. The system of claim 1, wherein the sensor system comprises at least one flow sensor for detecting an insufflation gas flow rate associated with at least one of the first and second insufflation gas inlets, and wherein the control system controls the valve system to actuate the at least one valve until the insufflation gas flow rate is sufficiently reduced.

6. The system of claim 2, wherein the control system is configured to switch from insufflation gas supply via the first insufflation gas inlet to insufflation gas supply via the second insufflation gas inlet upon determining that the at least one pressure or the at least one flow rate is below a predetermined threshold.

7. The system of claim 2, wherein the control system is configured to provide a notification indicative of a depletion of at least one of the at least two insufflation gas supply tanks.

8. The system of claim 7, wherein the notification is provided on a display of the system.

9. The system of claim 7, wherein the control system is configured to transmit the notification to an external system via a network connection.

10. The system of claim 7, wherein the notification provides an indication of which of the at least two insufflation gas supply tanks is depleted.

11. The system of claim 1, wherein the valve system comprises a valve that comprises two inlets, and the valve automatically actuates based on a pressure differential between the two inlets.

12. The system of claim 1, wherein the system is portable.

13. The system of claim 1, wherein the outlet is configured for fluidly connecting to an insufflation gas inlet of an insufflator.

14. The system of claim 1, wherein the system is an insufflator.

15. A method for supplying insufflation gas for a surgical procedure comprising:
supplying insufflation gas from a first insufflation gas supply tank located in an operating room to a surgical field; and
automatically switching from supply by the first insufflation gas supply tank to supply by a second insufflation gas supply tank based on at least one of a reduction in pressure of the first insufflation gas supply tank and a reduction in flow rate from the first insufflation gas supply tank.

16. The method of claim 15, comprising providing a notification of a depletion of the first insufflation gas supply tank.

17. The method of claim 16, wherein the notification indicates which of the at least two insufflation gas supply tanks is depleted.

18. The method of claim 15, comprising monitoring a pressure of the first insufflation gas supply tank and automatically actuating a valve to switch the supply in response to determining that a pressure of the first insufflation gas supply tank is below a predetermined threshold.

19. The method of claim 15, wherein the supply is automatically switched by a valve that automatically actuates based on a pressure differential.

20. The method of claim 15, further comprising monitoring an amount of insufflation gas provided to the surgical field and detecting an insufflation gas supply leak by comparing the amount of insufflation gas provided to the surgical field to a drop in pressure of at least one of the first and second insufflation gas supply tanks.

21. An apparatus for cleaning a surgical scope, the apparatus comprising:
a sheath for removably receiving a tube of the surgical scope, the sheath comprising a wall defining a channel for receiving the tube and a conduit that defines a fluid flow path;
a nozzle located at a distal end of the distal portion of the wall and configured for directing a fluid flow across a lens of the surgical scope to clean the lens; and
a first inlet for connecting a gas supply for supplying a gas flow to the fluid flow path and a second inlet for connecting a liquid supply for supplying a liquid flow to the fluid flow path.

22. The apparatus of claim 21, further comprising a valve for fluidly connecting and disconnecting the first and second inlets, respectively, to the fluid flow path.

23. The apparatus of claim 22, wherein the valve actuates automatically based on a pressure differential between the first and second inlets.

24. A method for cleaning a surgical scope comprising:
inserting the surgical scope into a sheath of a surgical scope cleaner;
flowing a liquid through a conduit of the sheath and spraying a lens of the surgical scope with the liquid via a nozzle of the surgical scope cleaner; and
flowing a gas through the conduit of the sheath and blowing the lens of the surgical scope with the gas via the nozzle of the surgical scope cleaner to remove the liquid from the lens.

25. The method of claim 24, comprising closing a gas supply pathway while flowing the liquid through the conduit and closing a liquid supply pathway while flowing the gas through the conduit.

26. The method of claim 25, wherein the liquid and gas supply pathways automatically close and open based on a pressure differential between the liquid and gas supply pathways.
